(19) United States
(12) Patent Application Publication
(10) Pub. No.: US 2013/0217122 A1
(43) Pub. Date: Aug. 22, 2013

(54) Expansion of Interferon-Gamma-Producing T-Cells Using Glypican-3 Peptide Library

(71) Applicants: The Trustees of the University of Pennsylvania, (US); Government of the United States of America as Represented by the Secretary of the Department of Veteran, (US)

(72) Inventor: David E. Kaplan, Media, PA (US)

(73) Assignees: The Trustees of the University of Pennsylvania, Philadelphia, PA (US); Veteran, Washington, DC (US); Government of the United States of America as Represented by the Secretary of the Department of

(21) Appl. No.: 13/773,090

(22) Filed: Feb. 21, 2013

Related U.S. Application Data
(60) Provisional application No. 61/601,299, filed on Feb. 21, 2012.

(51) Int. Cl. C12N 5/0783; C07K 14/725

(52) U.S. Cl. CPC C12N 5/0636; C07K 14/7051
USPC 435/372.3; 536/23.5

(57) ABSTRACT

The present invention encompasses a method for expanding an antigen specific T cell from a population of cells. The method of the present invention comprises contacting a population of cells with an MHC restricted antigenic glypican-3 peptide. In some instances, the T cell is expanded using monocyte-derived dendritic cells and defined MHC restricted antigenic glypican-3 peptides. In one embodiment, high-affinity antigen-specific T-cell receptors (TCRs) can be cloned from the antigen specific T cell.

### CROSS-REFERENCE TO RELATED APPLICATION

This application claims priority to U.S. Provisional Application Ser. No. 61/601,299, filed Feb. 21, 2012, the contents of which are incorporated by reference herein in their entirety.

### BACKGROUND OF THE INVENTION

Hepatocellular carcinoma (HCC) is the fifth most common malignancy and third-leading cause of cancer death worldwide (Parkin, 2001, Lancet Oncol 2:533-543). Hepatocellular carcinoma most frequently develops in patients with cirrhosis related to chronic viral hepatitis (Perz, et al., 2006, J. Hepatol. 45:529-538) at an approximate rate of 4% per year, often prior to the development of other complications of cirrhosis or portal hypertension. In these individuals, HCC primarily causes morbidity and mortality due to acceleration of hepatic failure and portal hypertension rather than via metastatic spread. Usually asymptomatic until late stages, HCC most often presents at an advanced stage at which time treatment options are quite limited (Leykum, et al., 2007, Clin Gastroenterol Hepatol 5:508-512). For this reason, novel approaches including immunotherapy have been considered as a potential adjunctive therapy in advanced HCC.

There are several theoretical and empiric reasons to expect that hepatocellular carcinoma might be poorly immunogenic and thus a challenging target for tumor vaccines. First, the liver microenvironment exerts a toleragenic effect due to various properties of liver sinusoidal endothelial cells and Kupffer cells (Schurich, et al., 2010, J Immunol 184:4107-4114; Crispe, et al., 2006, Immunol Rev 213:101-118). Second, HCC arises primarily in a cirrhotic liver, an immunosuppressive environment associated with phagocyte and antigen-presenting cell dysfunction (Rajkovic, et al., 1986, Hepatology 6:252-262; Kakazu, et al., 2009, Hepatology 50:1936-1945). Third, chronic viral infections, such as hepatitis B and C, which underlie the antecedent cirrhosis, induce intrahepatic antigen-specific effector T-cell exhaustion as well as regulatory T-cell generation (Nakamoto, et al., 2008 Gastroenterology 134(7):1927-37; Ebinuma, et al., 2008, J Virol 82:5043-5053). Furthermore, there is mounting evidence that hepatocellular carcinomas may directly recruit regulatory cell populations, such as M2 macrophages, myeloid-derived suppressor cells, and regulatory T-cells, to suppress tumor-directed T-cell effector responses (Hoechst, et al., 2008, Gastroenterology 135:234-243; Takai, et al., 2009, Cancer Biol Ther 8:2329-2338). Not surprisingly, tumor-antigen specific T-cells in patients with HCC, when detectable, have generally exhibited restricted effector functions (Gehring, et al., 2009, Gastroenterology 137:682-690). Nonetheless, some studies suggest that the presence of T-cell responses against panels of tumor antigens in HCC may be associated with improved prognosis (Hiroishi, et al., 2010, J Gastroenterol 45:451-458). Furthermore, T-cell activation by cytokines (Lygidakis, et al., 1995, J. Interferon Cytokine Res 15:467-472) and/or by therapeutic embolization of tumor-lysate-pulsed dendritic cells directly into HCCs has resulted in partial tumor control (Palmer, et al., 2009, Hepatology 49:124-132), suggesting that tumor-specific T-cells in HCC could play a role in retarding tumor growth.

Glypican-3 (GPC3), a glycosylphosphatidylinositol-linked heparan-sulfate proteoglycan, has been identified as a highly specific, membrane-associated, tumor antigen found in 66-100% of HCC (Capurro, et al., 2003, Gastroenterology 125:89-97; Libbrecht, et al., 2006, Am J Surg Pathol 30:1405-1411) with little or no expression in non-tumorous cirrhotic liver tissue or other normal adult tissues (Abdul-Al, et al., 2008, Hum Pathol 39:209-212; Baumhoer, et al., 2008, Am. J. Clin Pathol 129:899-906). GPC3 fosters HCC growth by altering Wnt signaling (Song, et al., J. Biol. Chem. 280 (2005) 2116-2125), modulating growth factors such as IGF-2, BMP-7 and FGF-2 (Filmus, et al., 1999, Medicina (B Aires) 59:546), and possibly by playing a role in M2 macrophage recruitment (Takai, et al., 2009, Cancer Biol Ther 8:2329-2338). GPC3 may be cleaved from the surface of expressing hepatocytes, thereby entering the circulation to allow serological detection (Hippo, et al., 2004, Cancer Res 64:2418-2423). While several groups have reported the ability to expand glypican-3-specific T-cells in mice (Komori, et al., 2006, Clin Cancer Res 12:2689-2697; Motomura, et al., 2008, Int J Oncol 32:985-990; Nakatsura, et al., 2004, Clin Cancer Res 10:8630-8640) and from a small number of human subjects (Komori, et al., 2006, Clin Cancer Res 12:2689-2697; O'Beirne, et al., 2010, J Exp Clin Cancer Res 29:48), this work has focused primarily on expanding CD8⁺ T-cell using specific HLA-types and highly immunodominant epitopes (Komori, et al., 2006, Clin Cancer Res 12:2689-2697; Motomura, et al., 2008, Int J Oncol 32:985-990; O'Beirne, et al., 2010, J Exp Clin Cancer Res 29:48) limiting the applicability of findings to more heterogeneous populations. Such approaches have also precluded the examination of the potential important role of tumor antigen-reactive CD4⁺ T-cells (Alisa, et al. 2005, Clin Cancer Res 11:6686-6694; Witkowski, et al., 2011 Int J. Cancer. 129, 2171-2182). Furthermore, cytolytic capacity of expanded GPC3-specific cells remained fairly weak even after long-term in vitro expansion under optimized conditions (Komori, et al., 2006, Clin Cancer Res 12:2689-2697) suggesting that antigen-specific T-cells expanded from HCC patients remain dysfunctional.

Thus, there is a need in the art to expand and characterize glypican-3-specific T-cells in order to generate a therapeutic population of glypican-3-specific T-cells. The present invention provides this and other advantages as described further herein.

### SUMMARY OF THE INVENTION

The present invention provides a method of expanding an antigen specific T cell in a population of cells. In one embodiment, the method comprises isolating the population of cells from a human, contacting the population of cells with an MHC restricted antigenic glypican-3 peptide, thereby expanding an antigen specific T cell from the population of cells.

In one embodiment, the human is an HLA-A2⁺ healthy donor.

In one embodiment, the T cell is specific for glypican-3.

In one embodiment, the MHC restricted antigenic glypican-3 peptide comprises the sequence selected from the group consisting of SEQ ID NO: 2, SEQ ID NO: 3, SEQ ID NO: 4, SEQ ID NO: 5, and SEQ ID NO: 6.

In one embodiment, the population of cells comprises peripheral blood mononuclear cells (PBMCs).

The invention also provides an isolated antigen specific T cell generated by isolating a population of cells from a human, contacting the population of cells with an MHC restricted antigenic glypican-3 peptide, expanding an antigen specific T cell that is reactive to the glypican-3 peptide, and isolating the expanded antigen specific T cell using a multimer wherein the multimer comprises the glypican-3 peptide.

The invention also provides an isolated polynucleotide encoding a T cell receptor (TCR) that is derived from an antigen specific T cell, wherein the antigen specific T cell is produced by isolating a population of cells from a human, contacting the population of cells with an MHC restricted antigenic glypican-3 peptide, expanding an antigen specific T cell that is reactive to the glypican-3 peptide, and isolating the expanded antigen specific T cell using a pentamer wherein the pentamer comprises the glypican-3 peptide.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A though 1F, is a series of images depicting T-cell reactivity to glypican-3 and control antigens ex vivo in HCC patients and controls. IFNγ spot-forming units (SFU) per million (SFU/10⁶) PBMC in response to glypican-3 15 mer peptides (FIG. 1A), survivin 15 mer peptides (FIG. 1B), CEF 15 mer peptides (FIG. 1C), recombinant human glypican-3 (FIG. 1D), recombinant human survivin (FIG. 1E), and Candida albicans (FIG. 1F) are shown for healthy donors, non-cirrhotic viral hepatitis, non-tumor-bearing cirrhotics, and HCC patients. Reference line indicates 50 SFU/10⁶ PBMC and bars show log-normalized mean values. P-values are shown from ANOVA on log-normalized data.

FIG. 2, comprising FIGS. 2A through 2D, is a series of images depicting expansion of IFNγ+ T-cells with short-term in vitro expansion by Elispot. PBMC were stimulated with 1 mg/ml survivin or glypican-3 5 mer peptide pools for 8 days with rhIL-2 100 U/ml added on days 2 and 5. T-cell lines were re-stimulated with peptide pools in Elispot assays. Background was subtracted and results normalized to SFU/10⁶ PBMC. FIGS. 2A through 2C depict comparison of ex vivo and in vitro 15 mer peptide responses for non-cirrhotic patients (FIG. 2A), cirrhotic patients (FIG. 2B), and hepatocellular carcinoma patients (FIG. 2C). P-values were obtained by matched pair analyses. FIG. 2D is an image depicting the comparison of serum glypican-3 levels in patients with and without IFNγ⁺ glypican-3-specific T-cells greater than 500 SFU/10⁶ PBMC after expansion.

FIG. 3, comprising FIGS. 3A through 3G, is a series of images depicting cytokine and degranulation profile of 15 mer peptide short-term in vitro-expanded T-cells. FIG. 3A is an image of a stacked column chart comparing median increase in glypican-3-specific TNFα- and/or IFNγ-secreting CD8+ T-cells in restimulated (subtracting derived from unrestimulated control wells) for patients with (left) and without (right) detectable IFNγ Elispot response. FIG. 3B is an image of a stacked column chart comparing median increase in glypican-3-specific TNFα- and/or IFNγ-secreting CD4+ T-cells in restimulated for patients with (left) and without (right) detectable IFNγ Elispot response. FIG. 3C is an image showing per lymphoid analysis demonstrating relative contribution of CD4+ (black bars) and CD8+ (white bars) T-cells to IFNγ response. FIG. 3D is an image showing per lymphoid analysis demonstrating relative contribution of CD4+ (black bars) and CD8+ (white bars) T-cells to TNFα response. FIG. 3E is an image showing frequency per subset of CD4+ and CD8+ T-cell TNFα, IFNγ, and CD107a responses against glypican-3. FIG. 3F is an image of a stacked column chart comparing median increase in CEF-specific TNFα- and/or IFNγ-secreting CD8+ T-cells under identical expansion conditions. FIG. 3G is an image demonstrating the distribution of CD8+ (left) and CD4+ (right) T-cell TNFα, IFNγ, and CD107a responses against glypican-3.

FIG. 4, comprising FIGS. 4A through 4B, is a series of images depicting cytokine and degranulation profile of 15 mer peptide short-term in vitro-expanded T-cells. FIG. 4A is an image depicting a representative intracellular cytokine FACS plots showing CFSE dilution versus TNFα (upper) and TNFα vs. IFNγ (lower) for four HCC patients with glypican-3-specific TNFα responses after 1 week of in vitro expansion using 15 mer pooled peptides. FIG. 4B is an image depicting an example in which PBMC from HLA-A2+ HCC patient were expanded with 9-10 mer optimal peptides. Pentamer frequency ex vivo (top left) and after expansion in vitro (bottom left) for Sur1M2 and 5 GPC3 pentamers are shown. Ex vivo IFNγ Elispot (top right) shows no IFNγ⁺ responses against any glypican-3 peptide with lack of increase in IFNγ production by Elispot after restimulation with cognate antigen (bottom right).

FIG. 5, comprising FIGS. 5A and 5B, is a series of images depicting that impact of PD-L1 and CTLA-4 blockade on total T-cell IFNγ production by Elispot.

FIG. 5A is an image demonstrating IFNγ response (SFU/10⁶ PBMC) after 1 week of in vitro expansion with either DMSO control, glypican-3 15 mer peptides, survivin 15 mer peptides, or HCV NS3 15 mer peptides in the presence of either control Ig or anti-PD-L1 mAb. P-value shown for matched pair analysis on log-normalized data. FIG. 5B is an image demonstrating IFNγ response after in vitro expansion with either DMSO control, glypican-3 15 mer pooled peptides, survivin 15 mer peptides, or HCV NS3 15 mer pooled peptides in the presence of either control Ig or anti-CTLA-4 mAb.

FIG. 6, comprising FIG. 6A through 6C, is a series of images depicting the impact of PD-L1 and CTLA-4 blockade on CD8 T-cell proliferation and cytokine production by intracellular staining FIG. 6A is an image depicting the frequency of proliferated (CFSE^{lo} TNFα⁻IFNγ⁻ per CD8⁺ T-cell in presence of control Ig versus anti-PD-L1 and anti-CTLA-4 mAbs for 7 individual patients. FIG. 6B is an image depicting the percentage of IFNγ⁺ (left) and TNFα⁺ (right) T-cells per CD4+ cells in presence of control Ig versus anti-PD-L1 and anti-CTLA-4 mAbs (p by matched pair analysis). FIG. 6C is an image depicting an example of effect on expansion and cytokine production of pentamer-specific CD8⁺ T-cells in presence of control Ig, anti-PD-L1 and anti-CTLA-4 mAbs for four glypican-3 pentamers.

FIG. 7, comprising FIGS. 7A through 7C, is a series of images that demonstrate the capacity of 15 mer peptides containing predicted HLA-A2.1-restricted epitopes to bind HLA-A2.1 in cell based culture system. FIG. 7A is an image depicting the T2 Affinity Index (AI)=(geometric MFIpeptide-geometric MFIcontrol)/(geometric MFI control) for individual 15 mer peptides for Glypican-3 (SEQ ID NOs: 10-57) and Survivin (SEQ ID NOs: 58-79). FIG. 7B is an image depicting the comparison of 15 mer AI and AI for 9/10 mer optimum peptides (SEQ ID NOs. 80-95). FIG. 7C is an image depicting the correlation of AI of 15 mer and 9/10 mer optimum peptides using Pearson correlation.

FIG. 8, comprising FIGS. 8A through 8D, is a series of images demonstrating the expression of PD-1 on peripheral glypican-3-specific CD8 T-cells from patients with hepatocellular carcinoma. FIG. 8A is an image of Zebra plots showing ex vivo CD8+ pentamer+/CD8+ T-cells in 1 representative among 9 total HLA-A2⁺ HCC patients for 5 GPC3-specific pentamers, Sur1M2, influenza matrix, and hepatitis C NS3₁₀₇₃₋₁₀₈₁. FIG. 8B is an image depicting the comparison of geometric mean MFI of PD-1 for pentamer⁻ versus pentamer⁺ CD8⁺ T-cells for each GPC3 and control pentamer. P-values reflect matched pair comparisons on log-normalized values. FIG. 8C is an image of representative histograms of PD-1 intensity on CD8⁺ pentamer⁺ T-cells. FIG. 8D is an image depicting the distribution of geometric mean MFI for PD-1 for 6 HLA-A2+ HCC patients stained and acquired simultaneously. Comparisons of differences among pentamers were performed using ANOVA on log-normalized data and each pentamer was also individually compared to the influenza pentamer.

FIG. 9, comprising FIGS. 9A through 9D, is a series of images depicting expression of CTLA-4, and LAG-3 on peripheral glypican-3-specific CD8 T-cells from patients with hepatocellular carcinoma. FIGS. 9A and 9B depict the comparison of geometric mean MFI of CTLA-4 (FIG. 9A) and LAG-3 (FIG. 9B) for pentamer⁻ versus pentamer⁺ CD8⁺ T-cells for each GPC3 and control pentamer. P-values reflect matched pair comparisons on log-normalized values. FIGS. 9C and 9D depict the distribution of geometric mean MFI for CTLA-4 (FIG. 9C) and LAG-3 (FIG. 9D) for 6 HLA-A2+ HCC patients stained and acquired simultaneously. Comparisons of differences among pentamers were performed using ANOVA on log-normalized data and each pentamer was also individually compared to the influenza pentamer.

FIG. 10 is an image demonstrating that low affinity HLA-A2-restricted T-cells circulate in peripheral blood in HCC patients and can be expanded in vitro. PBMC from HLA-A2+ HCC donor were cultured for 7 days in the presence of 15 mer overlapping peptides for glypican-3 plus optimal HLA-A2 epitopes and supplemented with rhIL-2. Influenza matrix peptide was used as positive control. Pentamer frequency is shown ex vivo and after in vitro simulation and results are representative of experiments from 3 HCC patients.

FIG. 11 is an image demonstrating that HLA-A2-restricted T-cells circulate in peripheral blood in normal donors. PBMC from 4 HLA-A2+ donors without liver disease were directly stained using HLA-2.1-glypican-3 pentamers and controls. Boxes indicate pentamer+ CD8 T-cell frequency.

FIG. 12, comprising FIGS. 12A and 12B, is a series of images demonstrating that multifunctional tumor-antigen-specific T-cells can be expanded from peripheral blood in normal donors as well as HCC patients. FIG. 12A is an image demonstrating that PBMC from 19 HCC and 5 HD were tested for IFNγ responses by Elispot ex vivo then expanded in vitro for 7 days with 15 mer overlapping peptide libraries for glypican-3 (top left) and survivin (top right), then retested by Elispot. Results are expressed as IFNγ spot-forming units (SFU) per million cells. FIG. 12B is an image depicting two examples of expanded glypican-3-specific CD8+ T-cells in healthy donors showing TNFα+CD107a+ (NTC028) or TNFα+IFNγ+ (NTC019) cells.

FIG. 13 is an image depicting activation markers on FAST-DC generated from cirrhotic patient. CD11c+ monocytes derived from PBMC were cultured 24 h with IL-4 and GM-CSF then matured with GM-CSF, IL-4, TNFα, IL-10, IL-6 and PGE₂ for 24 hours. FAST-DC expressed high levels of CD40, CD70, CD83, CD86, and HLA-DR (black line). Isotype control is shown in grey.

### DETAILED DESCRIPTION

The present invention includes compositions and methods for expanding a tumor antigen-specific T-cell. Preferably, the tumor antigen-specific T cell can be isolated and expanded from healthy and cirrhotic donors in HLA-independent fashion using antigen-presenting cells. Accordingly, the invention provides peptides for expanding antigen specific T cells.

In one embodiment, the invention provides peptide epitopes of glypican-3 antigen which are capable of being presented in conjunction with an MHC class I or a class II molecule such that they may be specifically recognized by a T cell. Accordingly, in a further aspect of the invention, peptide-specific T-cells can be detected or isolated using an MHC multimer, tetramer or a pentamer, comprising at least one of the peptide epitopes as described herein.

In one embodiment, the invention provides an antigen-presenting cell loaded with an epitope of the invention, wherein the epitope is presented on the antigen-presenting cell. The epitope can be bound to an HLA molecule on the antigen-presenting cell, whereby when an HLA-restricted cytotoxic lymphocyte (CTL) is present, a receptor of the CTL binds to a complex of the HLA molecule and the epitope. The antigen presenting cell can be a dendritic cell.

In another embodiment, the present invention provides novel cloned high-affinity HLA-A2-restricted tumor antigen-specific T-cell receptors (TCRs) derived from healthy or cirrhotic donors.

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

An “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides, and particularly at the carboxy- or amino-terminus, can be modified by methylation, amidation, acetylation or substitution with other chemical groups which can change a peptide's circulating half-life without adversely affecting activity of the peptide. Additionally, a disulfide linkage may be present or absent in the peptides.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “autologous” is meant to refer to any material derived from the same individual to whom it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

“Encoding” refers to the inherent property of specific sequences of nucleotides in a polynucleotide, such as a gene, a cDNA, or an mRNA, to serve as a template for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

As used herein, the term “fragment,” as applied to a protein or peptide, refers to a subsequence of a larger protein or peptide. A “fragment” of a protein or peptide can be at least about 20 amino acids in length; for example at least about 50 amino acids in length; at least about 100 amino acids in length, at least about 200 amino acids in length, at least about 300 amino acids in length, and at least about 400 amino acids in length (and any integer value in between).

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 5′-ATTGCC-3′ and 5′-TATGGC-3′ share 50% homology.

The term “immunoglobulin” or “Ig”, as used herein is defined as a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the term “modulate” is meant to refer to any change in biological state, i.e. increasing, decreasing, and the like.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

The term “subject” is intended to include living organisms in which an immune response can be elicited (e.g., mammals).

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based on the discovery that antigen-specific T cells can be isolated from a healthy donor compared to those isolated from an otherwise identical diseased donor. Preferably, the antigen specific T cell is reactive to glypican-3.

In one embodiment, the invention provides for compositions and methods to expand tumor antigen-specific T-cells. In some instances, the T cells can be expanded using optimized antigen-presenting cells, preferably dendritic cells (DCs).

In another embodiment, the invention relates to the identification of high-affinity MHC-restricted tumor antigen-specific T-cell receptors (TCRs). For example, allorestricted T-cell lines with specificity for a glypican derived peptide can be generated using peptide-pulsed cells. The TCRs from the T-cell lines that exhibit high peptide specificity and tumor reactivity while exhibiting low alloreactivity can be cloned. The cloned TCRs present a promising tool for the development of specific adoptive T-cell therapies to treat glypican overexpressing cancers. Preferably, the glypican overexpression cancer is hepatocellular carcinoma.

### T Cell Epitope Peptide

A T cell epitope of the invention is a short peptide that can be derived from a protein antigen. Antigen presenting cells can directly bind antigen via surface MHC molecules and/or internalize antigen and process it into short fragments which are capable of binding MHC molecules. The specificity of peptide binding to the MHC depends on specific interactions between the peptide and the peptide-binding groove of the particular MHC molecule.

Peptides which bind to MHC class 1 molecules are usually between 6 and 12, more usually between 8 and 12 amino or 8 and 10 amino acids in length. Typically, peptides are 9 amino acids in length. The amino-terminal amine group of the peptide makes contact with an invariant site at one end of the peptide groove, and the carboxylate group at the carboxy terminus binds to an invariant site at the other end of the groove. Thus, typically, such peptides have a hydrophobic or basic carboxy terminus and an absence of proline in the extreme amino terminus. The peptide is in an extended confirmation along the groove with further contacts between main-chain atoms and conserved amino acid side chains that line the groove. Variations in peptide length are accommodated by a kinking in the peptide backbone, often at proline or glycine residues. Peptides which bind to MHC class II molecules are usually at least 10 amino acids, for example about 13-18 amino acids in length, and can be much longer. These peptides lie in an extended confirmation along the MHC II peptide-binding groove which is open at both ends. The peptide is held in place mainly by main-chain atom contacts with conserved residues that line the peptide-binding groove.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. For example, the peptide may be obtained by cleavage from full-length glypican-3 protein. The composition of a peptide may be confirmed by amino acid analysis or sequencing.

The peptides of the invention can be tested in an antigen presentation system which comprises antigen presenting cells and T cells. For example, the antigen presentation system may be a murine splenocyte preparation, a preparation of human cells from tonsil or PBMC. Alternatively, the antigen presentation system may comprise a particular T cell line/clone and/or a particular antigen presenting cell type.

T cell activation may be measured via T cell proliferation (for example using ³H-thymidine incorporation) or cytokine production. Activation of TH1-type CD4+ T cells can, for example be detected via IFNγ production which may be detected by standard techniques, such as an ELISPOT assay.

Measurement of antigen-specific T cells during an immune response are important parameters in vaccine development, autologous cancer therapy, transplantation, infectious diseases, inflammation, autoimmunity, toxicity studies, and the like. Peptide libraries are crucial reagents in monitoring of antigen-specific T cells. The present invention provides improved methods for the use of a peptide library in analysis of T cells in samples including diagnostic, prognostic and immune monitoring methods. Furthermore the use of a peptide library in anti-tumor therapy are described elsewhere herein, including isolation of antigen-specific T cells capable of inactivation or elimination of undesirable target cells or isolation of specific T cells capable of regulation of other immune cells. The present invention also relates to MHC multimers comprising one or more tumor derived peptides.

In one embodiment, the present inventors have identified a number of epitopes of glypican-3. The identification of particular antigenic peptides provides new opportunities for the development of diagnostic and therapeutic strategies against cancer. In particular, the invention provides peptide epitopes of glypican-3 antigen which are capable of being presented in conjunction with an MHC class I or a class II molecule such that they may be specifically recognized by a T cell.

Advantageously, identification of novel T cell epitopes enable the production of MHC class I and class II multimers, tetramers and pentamers, useful as analytical tools delivering both increased sensitivity of immuno-monitoring and the ability to stain glypican-3 reactive T-cells in tumor biopsy samples. In addition, the detection of glypican-3 specific CTL in the periphery of individuals at risk of disease recurrence is a useful diagnostic tool.

Accordingly, in a further aspect, the invention provides an MHC multimer, tetramer or a pentamer comprising at least one of the MHC class I or II glypican-3 peptide epitopes as described herein.

The invention also provides compositions and methods for identifying peptides useful for cancer therapy. Preferably, the peptides are derived from glypican-3. Peptide sequences from a candidate protein (e.g., glypican-3) predicted to bind to HLA-A*0201 can be identified by a computer algorithm. Peptides are selected for synthesis according to predicted affinity with a cut-off value of 500 nM or less, but also higher values may be chosen. Peptides are synthesized and binding to HLA-A*0201 can be confirmed using biochemical assays. Peptide binding is compared with the binding achieved with a pass/fail control peptide, designated 100%, and with a positive control peptide. Corresponding HLA-A*0201-peptide multimers are also synthesized for peptides with a binding affinity above the pass/fail control peptide. These peptides are tested for the ability to generate a T cell line specifically reacting with the specific peptide-HLA-A*0201 complex. The cell line can be referred to as multimer-, tetramer-, or pentamer-positive T cells. Multimer positive cells indicate a high immunogenicity for the corresponding peptide. Additional responses can be measured to assess production of the cytokine interferon-γ, degranulation and killing of target cells.

In some embodiments, the peptides of the invention can be administered directly to a patient as a vaccine. Thus the peptides of the invention are immunogenic epitopes of specific proteins and are used in order to elicit a T-cell response to their respective proteins. In some embodiments, the polypeptide of the invention is administered directly to a patient as a vaccine. For example, in a patient that has leukemia, a polypeptide comprising a peptide from a hematopoietic cell specific protein is administered to the patient in order to elicit a T-cell response to the protein. The T-cell response leads to death of hematopoietic cells, including the cancerous cells, but is specific to these cells and does not result in an immune response to other cell types.

It is also to be noted that, in many patients, directly administering such a peptide will not elicit a T-cell response because the cell specific protein is a “self-protein” and any T-cells that are capable of binding the polypeptide when presented on an MHC molecule of the HLA alleles of the patient are tolerized. That is to say T-cells that would be reactive are either destroyed in the thymus of the patient during the selection process or are inactivated through central or peripheral tolerance mechanisms. Therefore, it is preferred that the peptides of the invention are used to generate T-cells obtained, or obtainable, from an allogeneic donor individual. This individual should preferably be HLA negative for an HLA allele of which the patient is HLA positive. For example, if the patient is HLA positive for the HLA allele HLA-A*0201 then T-cells are obtained from an individual who is negative for HLA-A*0201. It is generally preferred that the donor individual is otherwise HLA-identical to the patient. Antigen presenting cells (APCs) are then provided which display MHC molecules of the HLA-A*0201 allele and which are loaded with the peptide. The T-cells of the donor individual are then primed with the APCs and the resulting cells are allowed to proliferate.

The proliferated T-cells which are capable of binding the peptide of the invention when in complex with the HLA-A*0201 antigen are then enriched using artificial structures which comprise a plurality of peptide-MHC molecules (e.g. pentamers or tetramers). The T cells specific for the particular peptide-HLA-A*0201 complex within the mixture of T cells have the capacity to bind to these structures when mixed with them. The T cells are subsequently mixed with magnetic beads with the capacity to bind the artificial structures. The artificial structures and the T cells bound to them are then removed from the remainder of the mixture by magnetic attraction of the beads.

### Pulsed Cells

The present invention also provides cells pulsed with peptides of the invention. Preferably the cells to be pulsed are capable of expressing MHC class I or class II. MHC class 1 molecules can be expressed on nearly all cell types, but expression of MHC class II molecules is limited to so-called “professional” antigen presenting cells (APCs); B cells, dendritic cells and macrophages. However, expression of MHC class II can be induced on other cell types by treating with IFNγ.

Expression of MHC class I or MHC class II molecules can also be achieved by genetic engineering (i.e. provision of a gene encoding the relevant MHC molecule to the cell to be pulsed). This approach has the advantage that an appropriate MHC haplotype(s) can be chosen which bind specifically to the peptide(s).

Preferably the cell to be pulsed is an antigen presenting cell, i.e. a cell which, in a normal immune response, is capable of processing an antigen and presenting it at the cell surface in conjunction with an MHC molecule. Antigen presenting cells include B cells, macrophages and dendritic cells. In an especially preferred embodiment, the cell is a dendritic cell.

Preferably the cell is capable of expressing an MHC molecule which binds a peptide according to an aspect of the invention in its peptide binding groove. For example, the cell may express one of the following HLA restriction elements: B7, B8, Cw7 A1, A2 or A3 (for MHC class I).

Peptide pulsing protocols are known in the art (see for example Redchenko and Rickinson (1999) J. Virol. 334-342; Nestle et al (1998) Nat. Med. 4 328-332; Tjandrawan et al (1998) J. Immunotherapy 21 149-157). For example, in a standard protocol for loading dendritic cells with peptides, cells are incubated with peptide at 50 μg/ml with 3 μg/ml β-2 microglobulin for two hours in serum free medium. The unbound peptide is then washed off.

The pulsed cell of the present invention may be used as a vaccine, for example to stimulate a prophylactic or therapeutic anti-glypican immune response.

The present invention therefore also provides a method for treating and/or preventing a disease which comprises the step of administering a peptide-pulsed cell to a subject in need of same. Accordingly, the invention provides compositions comprising APC (e.g., DC) pulsed with a peptide of the invention. In this aspect, the DC can be considered as a vaccine.

In one embodiment, the vaccine composition in accordance with the invention comprises ex vivo administration of an epitope or cocktail of epitope-bearing peptides to peripheral blood mononuclear cells (PBMCs), or isolated DC therefrom, from the patient's blood or donor's blood. After pulsing the DC with peptides but prior infusion into the recipient, the DC is washed to remove unbound peptides. In this embodiment, a vaccine comprises peptide-pulsed DCs which present the pulsed peptide epitopes in HLA molecules on their surfaces.

The vaccines of the invention can include autologous dendritic cells. In alternative embodiments, the vaccines can include allogeneic dendritic cells. Dendritic cells suitable for use in the vaccination methods disclosed herein can be isolated or obtained from any tissue in which such cells are found, or can be otherwise cultured and provided. Dendritic cells can be found in, for example, but in no way limited to, the bone marrow, PBMCs of a mammal, or the spleen of a mammal. Additionally, any suitable media that promote the growth of dendritic cells can be used in accordance with the present invention, and can be readily ascertained by one skilled in the art.

The dendritic cells in the vaccines described herein can be pulsed with any desired antigen (i.e., incubated for a sufficient time to allow uptake and presentation of peptides of the antigens on MHC molecules) or epitopes of the antigens (e.g., peptide epitopes 7-25 amino acids in length). The epitopes are, for example, peptides 7 to 13 (e.g., 8 to 10, e.g., 9) amino acids in length.

The dendritic cells present epitopes corresponding to the antigens at a higher average density than epitopes present on dendritic cells exposed to a tumor lysate (e.g., a neural tumor lysate) (e.g., at a density that is at least 5%, 10%, 25%, 50%, 100%, or 200% higher). The dendritic cells can acquire the antigens or portions thereof (e.g., peptide epitopes) by incubation with the antigens in vitro (e.g., wherein cells acquire antigens by incubation with the combination of the antigens simultaneously, or with a subset of antigens, e.g., in separate pools of cells). In some embodiments, the dendritic cells are incubated with a composition including the peptides, wherein the peptides are synthetic peptides and/or were isolated or purified prior to incubation with the cells. In some embodiments, dendritic cells are engineered to express the peptides by recombinant means (e.g., by introduction of a nucleic acid that encodes the full length antigen or a portion thereof, e.g., the peptide epitope).

The dendritic cells of the invention can be used to activate antigen specific T cells. In turn, the antigen specific T cell of the invention and/or T cells expanded using the methods of the present invention, can be administered to an animal, preferably a human. When the T cells expanded using the methods of the invention are administered, the amount of cells administered can range from about 1 million cells to about 300 billion. The precise dosage administered will vary depending upon any number of factors, including but not limited to, the type of animal and type of disease state being treated, the age of the animal and the route of administration.

The antigen specific T cell can be administered to an animal as frequently as several times daily or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

An antigen specific T cell may be co-administered with the various other compounds (cytokines, chemotherapeutic and/or antiviral drugs, among many others). Alternatively, the compound(s) may be administered an hour, a day, a week, a month, or even more, in advance of an antigen specific T cell, or any permutation thereof. Further, the compound(s) may be administered an hour, a day, a week, or even more, after administration of an antigen specific T cell, or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds and the antigen specific T cell, and the like.

### T Cell Receptors

In addition to their role in combating infections, T cells have also been implicated in the destruction of cancerous cells. Autoimmune disorders have also been linked to antigen-specific T cell attack against various parts of the body. One of the major problems hampering the understanding of and intervention on the mechanisms involved in these disorders is the difficulty in identifying T cells specific for the antigen to be studied.

T cell receptors (TCRs) are closely related to antibody molecules in structure, and they are involved in antigen binding although, unlike antibodies, they do not recognize free antigen; instead, they bind antigen fragments which are bound and presented by antigen-presenting molecules. An important group of antigen-presenting molecules are the MHC class I and class II molecules that present antigenic peptides and protein fragments to T cells.

Variability in the antigen binding site of a TCR is created in a fashion similar to the antigen binding site of antibodies, and also provides specificity for a vast number of different antigens. Diversity occurs in the complementarity determining regions (CDRs) in the N-terminal domains of the disulfide-linked alpha (α) and beta (β), or gamma (γ) and delta (A), polypeptides of the TCR. The CDR loops are clustered together to form an MHC-antigen-binding site analogous to the antigen-binding site of antibodies, although in TCRs, the various chains each contain two additional hypervariable loops as compared to antibodies. TCR diversity for specific antigens is also directly related to the MHC molecule on the APC's surface to which the antigen is bound and presented to the TCR.

In the embodiments described elsewhere herein, a peptide of the invention can be located within the MHC molecule of a dendritic cell in order to generate suitable T-cells. In some embodiments, the MHC molecule is loaded with the peptide extracellularly by incubating cells at 37° C., 5% CO₂ for 4 hours with varying concentrations of peptide, then washed once in serum-free RPMI. However, in alternative embodiments, the antigen presenting cells are transfected with a polynucleotide encoding a fusion protein comprising the peptide connected to at least an MHC Class 1 molecule alpha chain by a flexible linker peptide. Thus, when expressed, the fusion protein results in the peptide occupying the MHC Class I binding groove. Suitable MHC Class 1 molecules and costimulatory molecules are available from public databases. Further details of the synthesis of such a fusion molecule may be found in Mottez et al, J Exp Med. 1995 Feb. 1; 181(2):493-502, which is incorporated herein by reference. The advantage of expressing a fusion protein of the peptide and the MHC molecule is that a much higher concentration of peptide is displayed on the surface of the antigen presenting cells.

In some situations in the preparation of T-cells, it is preferred that there is an HLA match between the antigen presenting cells and the T-cells. That is to say the antigen-presenting cells display an MHC molecule of an allele for which the donor of the T-cells is HLA positive. In some embodiments, this is achieved by obtaining the antigen presenting cells from a first individual and the T-cells from a second individual wherein the first and second individuals have an HLA match.

However, in alternative embodiments, the antigen presenting cells and the T-cells are obtained from the same individual but the antigen presenting cells are transfected with polynucleotides encoding the MHC molecule of a similar HLA allele. In some embodiments, the polynucleotide encodes a protein which encodes the MHC molecule connected to the peptide via a linker. There are numerous HLA Class I alleles in humans and the MHC molecule displayed by the antigen presenting cells, may, in principle, be of any of these alleles. However, since the HLA-A*0201 allele is particularly prevalent, it is preferred that the MHC molecule be of this allele. However, any HLA-A2 allele is usable or other alleles such as HLA-A1, HLA-A3, HLA-A 11 and HLA-A24 may be used instead.

Antigen presenting cells may be transfected as follows. A linearized DNA vector encoding the MHC molecule is transcribed in vitro to synthesize mRNA. Harvested APCs are then mixed with the mRNA and the cells are electroporated. Achievement of the appropriate level of transfection is controlled by staining with anti-MHC antibody and detection using flow cytometry.

In further embodiments of the present invention there is provided a method for preparing T-cells suitable for delivery to a patient suffering from cancer. The method comprises providing dendritic cells expressing an HLA molecule of a first HLA allele and locating a peptide in the binding groove of the HLA molecule. The peptide may or may not be a peptide of the invention. T-cells are then primed with the dendritic cells, the T-cells being obtained or obtainable from an individual who is HLA matched for a first HLA allele. As described elsewhere herein, the dendritic cells may either be obtained from a first donor individual and the T-cells from a second donor individual wherein the first and second donor individuals are HLA matched. The advantage of using a dendritic cell, rather than a non-professional antigen presenting cell is that it results in a much higher stimulus of the T-cells

In these embodiments, it is preferred that the peptide is a cell type specific peptide, that is to say a peptide that is obtained from a protein which is only expressed, or is expressed at a much higher level (e.g. at least 10× higher concentration) in specific cells than in other cell types.

The T-cells prepared in accordance with the invention are administered to patients in order to treat cancer in the patients. In principle, the T-cells of the invention are capable of being used for the treatment of many different types of cancer including leukemia, lymphomas such as non-Hodgkin lymphoma, multiple myeloma and the like.

Thus, in some embodiments of the present invention, pharmaceutical preparations are provided comprising a T-cell of the invention and a pharmaceutically acceptable carrier, diluent or excipient, further details of which may be found in Remmington's Pharmaceutical Sciences in US Pharmacopeia, 1984 Mack Publishing Company, Easton, Pa., USA.

As discussed elsewhere herein, the HLA allele of the MHC molecule used to present the peptide to the T-cells is an HLA allele also expressed by the patient and therefore when the T-cells are administered to the patient, they recognize the peptide displayed on MHC molecules of that HLA allele.

In some alternative embodiments, multiple sets (e.g. 2 or 3 sets) of T-cells are provided, each T-cell being specific for a different peptide. In each case, the T-cells are allogeneic, as described elsewhere herein, that is to say the HLA allele of the MHC molecule on which the peptide is displayed during preparation of the T-cells is an HLA allele which is not expressed in the donor individual from whom the T-cells are obtained. The peptides may all be from the same cell specific protein or may be from different proteins but specific for the same cell type. The peptide may or may not be a peptide of the invention. In some embodiments, the multiple sets of T-cells are administered simultaneously but in other embodiments they are administered sequentially.

Reference has been made to the preparation and provision of T-cells. However, it is to be appreciated that the important feature of the T-cells is the T-cell receptor (TCR) which is displayed on the T-cells and, more specifically, the specificity of the T-cell receptor for the complex of the peptide and the MHC molecule. Therefore, in some alternative embodiments of the invention, following the preparation of T-cells as described elsewhere herein, the T-cell receptors of T-cells specific for a certain peptide when complexed with an MHC molecule of a particular allele are harvested and sequenced. A cDNA sequence encoding the T-cell receptor is then generated and which can be used to express the T-cell receptor recombinantly in a T-cell (e.g. the patient's own T-cells or T-cells from a donor). For example, the cDNA may be incorporated into a vector such as a viral vector (e.g. a retroviral vector), lentiviral vector, adenoviral vector or a vaccinia vector. Alternatively, a non-viral approach may be followed such as using naked DNA or lipoplexes and polyplexes or mRNA in order to transfect a T-cell.

Thus a T-cell which is “obtainable” from a donor individual includes a T-cell which is obtained recombinantly in the manner described elsewhere herein because the recombinantly expressed TCR is naturally produced.

Since transfected T-cells also display their endogenous TCRs, it is preferred that the T-cells are pre-selected, prior to transfection, to eliminate T-cells that would give rise to graft-versus-host disease. In some embodiments, the T-cells are pre-selected such that the specificity of their endogenous TCRs is known. For instance, T-cells are selected which are reactive with glypican-3. In other embodiments, the T-cells are obtained from the patient and thus are naturally tolerized for the patient. This approach can only be adopted where the T-cells of the patient are healthy.

In some alternative embodiments, the T-cell receptor, as a whole, is not recombinantly expressed but rather the regions of the T-cell receptor which are responsible for its binding specificity are incorporated into a structure which maintains the confirmation of these regions. More specifically, complementarity determining regions (CDRs) 1 to 3 of the T-cell receptor are sequenced and these sequences are maintained in the same conformation in the recombinant protein.

Therefore, in some embodiments of the invention, only the T-cell receptors, or a polynucleotide encoding the T-cell receptors are provided. For example, in one specific embodiment, allo-restricted T-cell lines which are reactive to a particular peptide from a protein, such as glypican-3, when displayed by an MHC molecule of the HLA allele HLA-A*0201 are generated as described elsewhere herein. The T cell lines are cloned, and the T-cell receptors from one or several T-cell clones are isolated and sequenced. A cDNA encoding the T-cell receptor is then prepared and inserted into an expression cassette or vector. When a patient who is HLA-A*0201 positive and suffering from cancer requires treatment, T-cells from a donor individual are transfected with the vector or expression cassette or mRNA and the T-cell receptors are expressed by and displayed on the T-cells. The T-cells are then administered to the patient in order to elicit a T-cell response to hematopoietic cells and eliminate them from the patient's body. If the T-cells are transfected with mRNA, the expression is transient and thus new hematopoietic cells of various lineages arise from the patient's own stem cells.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Expansion of Interferon-Gamma-Producing Multifunctional CD4+ T-Cells and Dysfunctional CD8+ T-Cells by Glypican-3 Peptide Library in Hepatocellular Carcinoma Patients

Glypican-3 is a promising target for immunotherapy for hepatocellular carcinoma, but limited data exist regarding its immunogenicity in patients with diverse HLA types, immunogenicity for CD4⁺ T-cells, and the impact of inhibitory co-stimulation on glypican-3-specific T-cells. Using a 15 mer overlapping peptide library for glypican-3, PBMC from patients with HCC were assessed ex vivo and after short-term in vitro expansion for tumor antigen-specific T-cell responses with and without blockade of PD-1/PD-L1 and CTLA-4 signaling. It was observed that glypican-3-specific T-cells were undetectable ex vivo, but primarily IFNγ⁺TNFα⁺ CD4⁺ T-cells expanded with short-term in vitro stimulation in 10/19 (52%) patients. Glypican-3-specific CD8⁺ T-cells predominantly produced TNFα, but did not secrete IFNγ nor degranulate. CTLA-4 and PD-1 blockade minimally impacted the cytokine secretion and proliferation of glypican-3-specific T-cells.

The materials and methods employed in these experiments are now described.

Patients

Subjects and controls were recruited from the Gastroenterology Clinics at the Philadelphia Veterans Affairs Medical Center following informed consent on an institutional review board-approved protocol. All patients were assessed for baseline demographics, hepatitis viral serologies, alcohol use history, and prior therapy for hepatocellular carcinoma. HCC patients were diagnosed histologically or via standard radiological and serological criteria (Bruix, et al., 2005, Hepatology 42:1208-1236). Controls included patients with hepatitis C-induced cirrhosis with no evidence of HCC by serial imaging and alpha-fetoprotein screening (cirrhotic group, CIR), hepatitis C patients with F1-2 fibrosis by biopsy within the preceding 5 years (early-stage viral hepatitis, EVH), and healthy donors (HD) with no evidence of chronic liver disease.

HLA Typing

In all patients, HLA-A2.1 expression was determined using flow cytometry with anti-HLA-A2.1 (clone BB7.2, BD Biosciences, Franklin Lakes N.J.). Results were confirmed with Terasaki HLA Class I Typing Tray (One Lambda, Canoga Park Calif.).

Peptides and Proteins

Libraries of 15 mer peptides offset by 6 and overlapping by 9 amino acids for the human glypican-3 (580aa, NP_{—}004475) and survivin (142aa, NP_{—}001159) were commercially synthesized (Proimmune, Oxford UK). Selected individual 9-10 mer peptides predicted to bind to human HLA-A2.1 based on online algorithms (BIMAS (Parker, et al., 1994, J Immunol 152:163-175), SYPEITHI (Rammensee, et al., 1999, Immunogenetics 50:213-219), and RankPep (Reche, et al., 2002, Hum Immunol 63:701-709) were also synthesized. Recombinant human glypican-3 (Gln 25-His 559) and survivin were obtained commercially (R&D Systems, Minneapolis Minn. and Genemega Inc., San Diego Calif. respectively). In previous studies using 15 mer peptide libraries for the hepatitis C virus, 15 mer peptides were shown to be immunogenic for both CD4⁺ and CD8⁺ T-cells (Sugimoto, et al., 2003, Hepatology 38:1437-1448). A mixture of CMV, EBV, and Influenza (CEF) 9-10 mer control peptides (Cellular Technology Ltd., Cleveland, Ohio) were used as positive controls for CD8⁺ effector T-cell responses.

Isolation of PBMC

Peripheral blood mononuclear cells (PBMC) were isolated using Ficoll-Histopaque (Sigma, St. Louis Mo.) density centrifugation. Cells were resuspended in RPMI 1640 with L-glutamine (Invitrogen) with 10% human AB serum (Sigma Inc., St. Louis, Mo.), 1.5% HEPES (Invitrogen) and 1% penicillin/streptomycin (Invitrogen).

In Vitro T-Cell Expansion

4×10⁶ PBMC were stimulated with media control, pooled glypican-3 15 mer peptides (95 peptides, 1 μg/ml each), or CEF peptide pool (1 μg/ml) for 8 days at 37° C. 5% CO₂ with rhIL-2 100 U/ml (Novartis/Chiron, Emeryville, Calif.) added on day 2 and day 5. Additional antigen-presenting cells (APC) were not utilized due to the presence of sufficient number of APCs in whole PBMC. For proliferation and blocking experiment, PBMC were labeled with 5 uM CFSE-DA (Molecular Probes, Eugene Oreg.) per manufacturer's instructions, then incubated with survivin-, glypican-3- or control peptide pools in the presence of NA/LE control Ig (Biolegend, San Diego Calif.), anti-PD-L1 (clone 29A.2E3, Biolegend) and/or anti-CTLA-4 (clone BNI3, BD Biosciences, San Jose Calif.) with rhIL-2 added on day 2 and day 5.

Flow Cytometry

All antibodies were purchased from Becton Dickinson (Becton Dickinson, Franklin Lakes, N.J.) except where specified. The cutoff for each marker was based on the isotype antibody. Data were acquired on a modified FACSCanto (BD), and analyzed using FlowJo (Tree Star Inc., Ashland Oreg.).

Intracellular Cytokine Staining

In vitro expanded cells were restimulated for 6 hours with survivin- or glypican-3-derived 15 mer peptide pools and controls in the presence of anti-CD 107a-PE and monensin, fixed and permeabilized using BD Cytoperm/Cytofix (BD), then stained intracellularly for IFNγ PE-Cy7 and TNFα APC (BD).

IFNγ Elispot

Antigen-specific T cell IFNγ responses were examined ex vivo and after in vitro expansion in cytokine Elispot assay as previously described (Kaplan, et al., 2008, J Hepatol 48: 903-913). In ex vivo assays, PBMC (2×10⁵/well) were plated in 96-well Elispot plates pre-coated with anti-IFNγ (5 μg/ml, Pierce, Rockford Ill.) and stimulated with peptide matrices for glypican-3 (95 peptides, 7×7×2 matrix) and human survivin (22 peptides, 5×5 matrix), pooled glypican-3 peptides (95 peptides), pooled surviving peptides (22 peptides) (1 μg/ml), recombinant glypican-3, and recombinant survivin proteins (10 mg/ml) in duplicates. Negative control wells without peptides (6 replicates) and positive control wells with 2 μg/ml phytohemagglutinin, 1 μg/ml CEF and 20 μg/ml C. albicans. After 8 days in vitro expansion, 5×10⁴ cells/well were stimulated with each peptide pool (1 μg/ml) in duplicates with positive (PHA) and negative controls (media, irrelevant peptide pool). Plates were analyzed for spot forming units (SFU), excluding assays with high background (average>10 SFU/well in negative control wells) or no response to positive control stimulation on an AID Elispot ELF04 (AID Diagnostika, Strassberg, Germany).

HLA-A2.1 Pentamer Staining

In HLA-A2+ patients, HCC-specific T-cells were detected ex vivo and after in vitro expansion using peptide-loaded HLA-A2.1 pentamers (Proimmune, Oxford UK) loaded with following 9-10 mer epitopes: Survivin₉₆₋₁₀₄ (LMLGEFLKL; SEQ ID NO: 1) (Sur1M2), Glypican-3₄₄₋₅₂ (RLQPGLKWV; SEQ ID NO: 2) (Komori, et al., 2006, Clin Cancer Res 12:2689-2697), Glypican-3₁₃₆₋₁₄₅ (SLTPQAFEFV; SEQ ID NO: 3), Glypican-3₁₄₂₋₁₅₁ (FEFVGEFFTD; SEQ ID NO: 4) (Komori, et al., 2006, Clin Cancer Res 12:2689-2697), Glypican-3₁₆₉₋₁₇₇ (ELFDSLFPV; SEQ ID NO: 5) (Komori, et al., 2006, Clin Cancer Res 12:2689-2697), Glypican-3₅₆₃₋₅₇₂ (KLLTSMAISV; SEQ ID NO: 6), HTLV-I tax₁₁₋₁₉ (LLFGYPVYV; SEQ ID NO: 7), HCV NS3₁₀₇₃₋₁₀₈₁ (CINGVCWTV; SEQ ID NO: 8) and Influenza Matrix₅₈₋₆₆ (GILGFTFVL; SEQ ID NO: 9). 1×10⁶ PBMC were stained with APC-labeled pentamer and the following antibodies (from BD, except as indicated): a dump channel containing anti-CD4 PerCP (RPA-T4), anti-CD14 PerCP (MΦP9), anti-CD19 PerCP (SJ25C1) and Viaprobe; anti-CD8 APC-H7 (SK1); anti-PD-1 FITC (MIH4); and biotinylated anti-LAG-3 (BAF2319, R&D Systems) detected with streptavidin-Alexa700 (Invitrogen). Cells were permeabilized and fixed using BD Cytoperm/Cytofix and stained intracellularly for anti-CD152 PE (CTLA-4, BNI3).

Statistical Analysis

The median values for clinical and immunologic parameters were compared using ANOVA, the nonparametric Kruskal-Wallis ANOVA, Wilcoxon Rank Sum or Mann-Whitney U test. The frequency of positive responses was compared using χ2 or Fisher's exact test based on sample size. Spearman rank correlation was used for bivariate correlation of variables. Multivariate regression was performed using JMP 7 (SAS Institute Inc, Cary N.C.). A p-value<0.05 was considered significant.

The results of the experiments are now described.

### Patient Characteristics

Thirty-three patients with hepatocellular carcinoma provided samples (Table 1). Median age was 57 years (range 49-67), 21 (63%) patients were black, and 12 (36%) were HLA-A2.1 positive. Except for one patient each with hepatitis B and non-alcoholic steatohepatitis, all patients were infected with hepatitis C virus and 25 had prior histories of alcohol dependence. Solitary tumors were present in 19/33 (58%, range 1-3 tumors) with a median size of the largest tumor being 3.8 cm (range 1.5-11.5 cm). 8/33 cases were confirmed histologically, the remainder confirmed using standard noninvasive criteria (Bruix, et al., 2005, Hepatology 42:1208-1236). The distribution of BCLC tumor stages were as follows: A 16; B 13; C 3; D 1. Five patients received transarterial chemoembolization prior to recruitment, but none within 3 months of enrollment. Serum AFP levels were >20 mg/dl in 22 patients (67%) and >200 mg/dl in 12/33 (36%). Serum glypican-3 was detectable in 22/33 (66.7%). Controls included 10 patients with HCV cirrhosis with no evidence of HCC, 6 HCV patients with F1-2, and 15 healthy donors with no evidence of chronic liver disease (Table 2). HCC patients were all male and modestly older than healthy donors, a group that included 5 women, but were well matched with respect to age, gender and ethnicity with cirrhotic and non-cirrhotic hepatitis C controls.

TABLE 1
Baseline characteristics of hepatocellular carcinoma patients.

| | | | | Underlying | | | Serum | Serum | |
|---|---|---|---|---|---|---|---|---|---|
| Patient | | | | liver | Biopsy | HLA- | AFP | glypican- | BCLC |
| ID | Age | Eth | Gnd | disease | confirmed? | A02 | mg/dI | 3 pg/mi | stage |
| HCCOO1 | 57 | W | M | HCV/EtOH | + | + | 58,700 | 219.2 | B |
| HCCOO2 | 57 | B | M | HCV/EtOH | + | + | 13 | <50 | A |
| HCCOO3 | 54 | B | M | HCV/EtOH | − | − | 1720 | <50 | A |
| HCCOO4 | 60 | W | M | HCV/EtOH | + | − | 23,700 | 1724.4 | B |
| HCCOO5 | 60 | B | M | HCV/EtOH | − | + | 12,600 | 820.7 | B |
| HCCOO6 | 49 | B | M | HCV/EtOH | − | − | 58 | 406.0 | A |
| HCCOO7 | 62 | B | M | HCV/EtOH | − | − | 421 | 1012.8 | B |
| HCCOO8 | 58 | B | M | HCV/EtOH | − | − | 94 | 213.5 | A |
| HCCOO9 | 61 | W | M | HCV/EtOH | − | − | 430 | 442.8 | B |
| HCCO1O | 53 | B | M | HCV/EtOH | − | − | 70 | 327.4 | A |
| HCCO11 | 63 | B | M | HCV/EtOH | + | + | 5 | 169.8 | B |
| HCCO12 | 61 | W | M | HCV | − | − | 3 | <50 | A |
| HCCO13 | 58 | B | M | HCV/EtOH | − | − | 17 | <50 | A |
| HCCO14 | 58 | B | M | HCV | − | − | 7230 | 253.2 | B |
| HCCO15 | 57 | B | M | HCV/EtOH | + | − | 24 | 206.0 | B |
| HCCO16 | 56 | B | M | HCV/EtOH | − | − | 4 | 310.3 | A |
| HCCO17 | 55 | B | M | HCV | − | + | 9 | <50 | A |
| HCCO18 | 52 | B | M | HCV | − | − | 118 | 163.6 | A |
| HCCO19 | 50 | B | M | HCV/EtOH | − | + | 1240 | 431.5 | C |
| HCCO21 | 59 | B | M | HCV/EtOH | + | + | 21,000 | <50 | B |
| HCCO22 | 59 | B | M | HCV | − | − | 50 | <50 | B |
| HCCO23 | 57 | W | M | HCV/EtOH | + | + | 1130 | 152.9 | B |
| HCCO24 | 67 | W | M | HCV | − | + | 12 | <50 | A |
| HCCO25 | 59 | W | M | NAFLD | + | − | 82 | <50 | A |
| HCCO26 | 56 | B | M | HCV/EtOH | − | − | 1150 | <50 | B |
| HCCO27 | 57 | W | M | HCV | − | − | 12 | 556.7 | A |
| HCCO28 | 56 | B | M | HCV/EtOH | − | + | 46 | 115.4 | A |
| HCCO29 | 58 | B | M | HCV/EtOH | − | − | 14 | 76.0 | D |
| HCCO3O | 57 | W | M | HCV/EtOH | − | − | 9 | <50 | A |
| HCCO31 | 62 | B | M | HCV/EtOH | − | − | 2110 | 68.2 | C |
| HCCO32 | 59 | W | M | HCV/EtOH | − | − | 53 | 850.1 | B |
| HCCO33 | 54 | W | M | HCV/EtOH | − | + | 119 | 3040 | C |
| HCCO34 | 56 | W | M | HCV/EtOH | − | + | 18 | 328.0 | A |

Ethnicity: W (White), B (Black).
Underlying liver diseases: HCV (hepatitis C). EtOH (alcoholic liver disease), NAFLD (non-alcoholic liver disease). HBV (hepatitis B).

TABLE 2
Demographics of hepatocellular carcinoma patients and controls.

| | HCC | CIR | EVH | HD | p |
|---|---|---|---|---|---|
| N | 33 | 10 | 6 | 15 | |
| Age (Median | 57 [49-67] | 55 [45-69] | 56 [44-64] | 49 [25-60] | 0.0026¹ |
| [Range]) | | | | | |
| Gender (M/F) | 33/0 | 10/0 | 6/0 | 10/5 | 0.0011² |
| Ethnicity | 12/20/1 | 5/5/0 | 2/4/0 | 10/4/1 | 0.2 |
| (W/B/H) | | | | | |
| Hepatitis C | 31 [94%] | 10 [100%] | 6 (100%] | 0 [0%] | |
| infected | | | | | |
| (N [%]) | | | | | |

¹HCC vs. HD p = 0.0001; HCC vs. CIR p = 0.049; and HCC vs. EVH p = 0.11.
²Difference entirely related HD group.

### IFNγ⁺ T-Cell Responses Against Glypican-3 Cannot be Detected Ex Vivo in HCC Patients or Controls

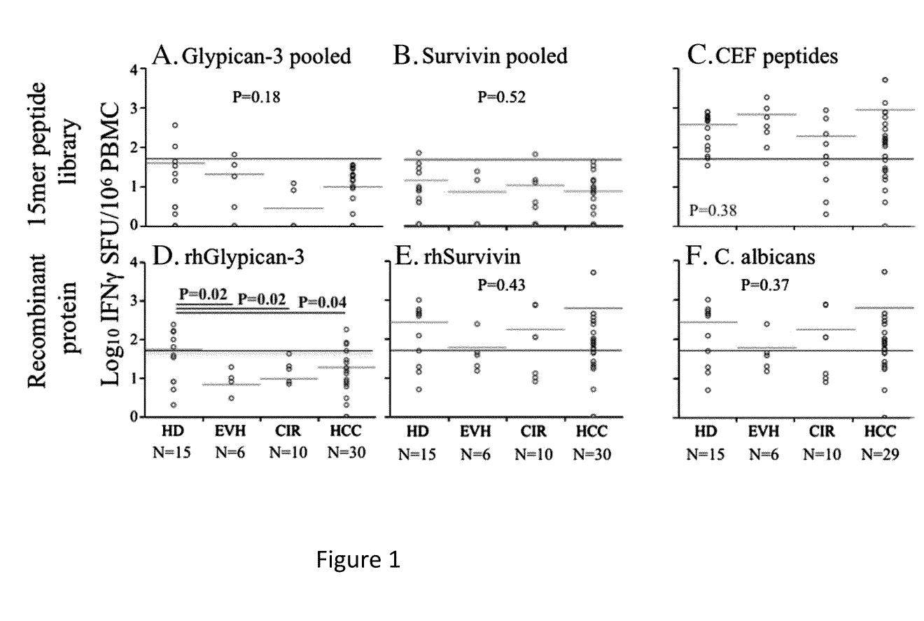

Utilizing 15 mer overlapping peptides in IFNγ Elispot, experiments were performed to quantify total T-cell responses against glypican-3 in patients with HCC and relevant controls. Using an arbitrary cutoff of 50 SFU/10⁶ PBMC (10 SFU/well above background) to define positive responses, no positive IFNγ responses in HCC patients against pooled glypican-3 15 mer peptides (FIG. 1A) nor against pooled survivin 15 mer peptides was observed (FIG. 1B). Positive responses occurred in fewer than 10% of non-HCC controls. Consistent with results for pooled peptides, peptide matrices revealed no convincing detectable T-cell reactivity to any individual glypican-3 15 mer peptide in any patient. The lack of detection of T-cell IFNγ⁺ response to the peptide libraries did not reflect global T-cell suppression as CD8⁺ T-cell reactivity to CEF viral peptides and CD4⁺ T-cell reactivity against C. albicans lysate remained detectable in the majority of cirrhotic patients (FIGS. 1C and 1F). A significant minority of healthy donor patients (5/15) but no EVH, CIR and only a small minority of HCC (4/30, only 1 of whom had detectable serum glypican-3 levels) had detectable CD4⁺ IFNγ⁺ responses against recombinant human glypican-3 (FIG. 1D), suggesting that endogenously expanded Tₕ1 effector T-cells against glypican-3 are relatively frequently detectable in health but become suppressed during the course of HCV disease. By contrast, CD4⁺ T-cell responses against human survivin were readily detected in most HCC patients as well as controls (FIG. 1E) indicating both the absence of a global CD4⁺ T-cell defect as well as differential regulation of Th1 responses against various tumor antigens. Overall however, these data indicate that CD4⁺ and CD8⁺ T-cells reactive to glypican-3 either circulate at extremely low frequencies or are highly suppressed in vivo in HCC patients.

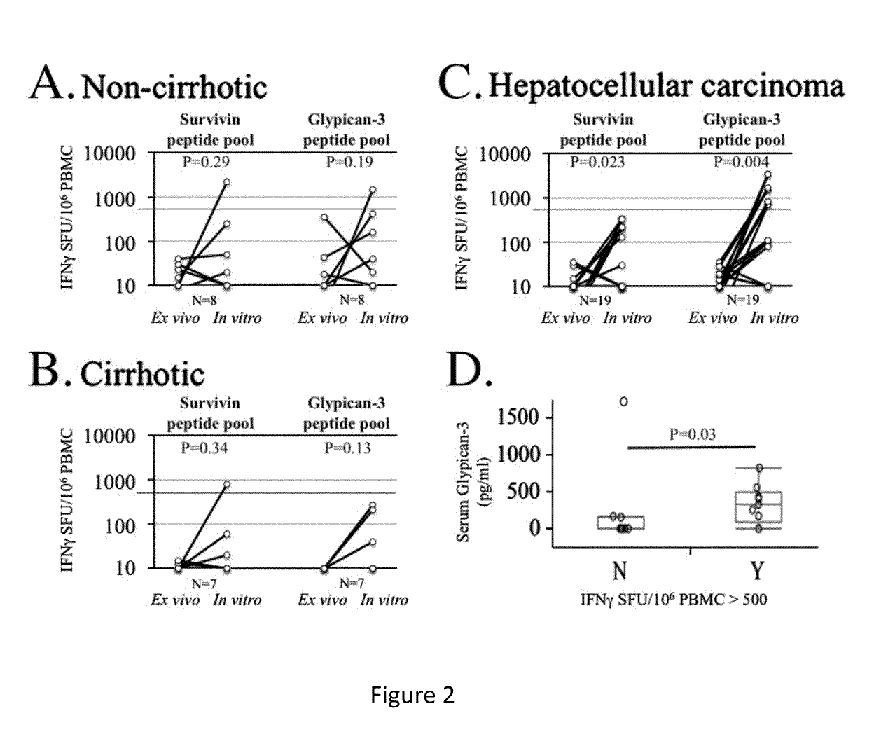

Glypican-3-Specific IFNγ⁺ Cells can be Expanded In Vitro from Nearly Half of HCC Patients

Where adequate lymphocytes were available, experiments were performed to expand freshly isolated PBMC with pooled 15 mer peptides for glypican-3 in the presence of rhIL-2 in vitro for a short term and then re-examined peptide-specific cytokine secretion by IFNγ Elispot and intracellular cytokine staining Unstimulated DMSO, survivin and CEF controls were used in all experiments. In all but 1 HCC patient, CEF control peptide stimulation resulted in expansion of antigen-specific IFNγ-secreting T-cells to greater than 1000 SFU/10⁶ PBMC reflecting the absence of a global CD8⁺ T-cell defect. Glypican-3-specific IFNγ-secreting T-cells could not be detected from PBMC expanded by rhIL-2 alone without peptides in any case. As shown in FIGS. 2A and 2B, detection of either glypican-3- or survivin-specific IFNγ⁺ T-cells in non-HCC patients to levels greater than 500 SFU/10⁶ PBMC (0.005%) with short-term in vitro expansion was a rare event, occurring in only one healthy control subject. By contrast, expansion of glypican-3-specific T-cells to greater than 500 SFU/10⁶ PBMC (0.005%) occurred in 10/19 (52%) of HCC patients (FIG. 2C). Pre- and post-expansion glypican-3-specific IFNγ SFU were markedly increased in HCC patients (mean 9 vs. 815 SFU/10⁶ PBMC, p=0.0038) but were not statistically increased for either the non-cirrhotic or cirrhotic groups or combination thereof (p=0.11). Interestingly, expansion of glypican-3-specific T-cells to greater than 500 SFU/10⁶ PBMC within the HCC group was associated with greater antigen burden, using serum glypican-3 levels as a surrogate marker of antigen expression (FIG. 2D, p=0.03), similar to recent findings correlating AFP-specific CD4⁺ T-cell IFNγ responses and serum AFP levels (Alisa, et al., 2005, Clin Cancer Res 11:6686-6694; Witkowski, et al., 2011 Int J. Cancer. 129, 2171-2182). Thus, among HCC patients, presence of tumor antigen was associated with in vivo expansion of antigen-specific T-cells at a precursor frequency significantly greater than present in healthy donors, non-cirrhotic liver disease and cirrhotic controls.

### Despite Expansion of Peripheral Glypican-3-Specific Th1 Cells, Glypican-3-Specific CD8+ T-Cells Exhibit Restricted Functional Responses

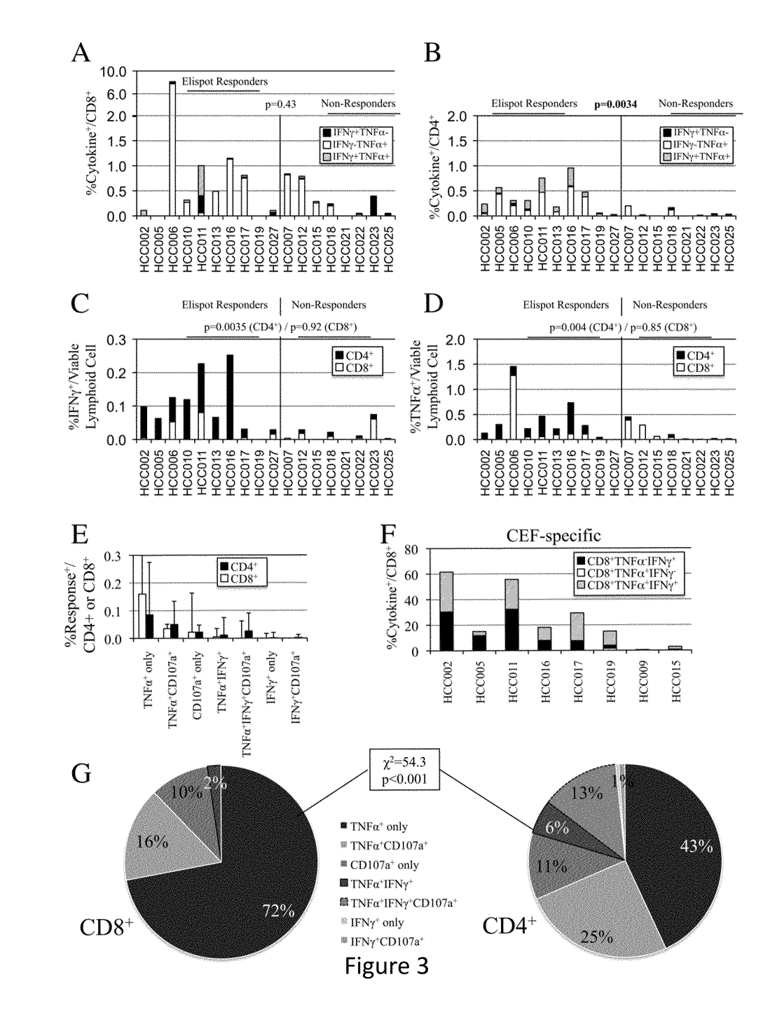

Peptide-expanded T-cells were restimulated on day 8 to measure CD107a uptake, a measure of degranulation, and production of IFNγ and TNFα by intracellular cytokine staining On a per subset level, the median cytokine response (IFNγ, TNFα, and/or CD107a) was 0.29% of CD4⁺ and 0.30% of CD8⁺ T-cells among HCC patients. Expansion of glypican-3-specific IFNγ-secreting CD8⁺ T-cells to greater than 0.25%/CD8⁺ T-cell was only seen in one normal donor and three of 18 HCC patients (median IFNγ 7CD8⁺ 0.03%, range 0-0.94%, FIG. 3A black plus grey bars). The predominant cytokine profile among CD8⁺ T-cells was a TNFα⁺IFNγ⁻ pattern (median of 0.13%/CD8+ T-cells) with 11/18 subjects having greater than 0.25% of CD8⁺ T-cell responding only by TNFα secretion (FIG. 3A). CD8⁺ T-cell cytokine secretion did not correlate with IFNγ Elispot results. While on a per subtype analysis, median IFNγ⁺ secretion per CD4⁺ T-cell was similar to CD8+ T-cells (median IFNγ⁺/CD4⁺ 0.03%, range 0-0.38%), there was a strong correlation between CD4+IFNγ⁺ frequency and IFNγ Elispot response (FIG. 3B). Reanalyzing these responses relative to all lymphoid cells (compensating for CD4:CD8 ratios), as shown in FIG. 3C, Elispot “responders” had significantly higher frequency of CD4⁺IFNγ⁺ (black bars, 0.069 vs. 0.003%, p=0.0035) but not CD8⁺IFNγ (white bars, 0.004 vs. 0.004%, p=0.92). CD4⁺TNFα⁺ responses were similarly more frequent in Elispot responders than nonresponders (median 0.167% vs. 0.046%, p=0.004) while no significant difference was seen for CD8⁺TNFα⁺ response (FIG. 3D). Glypican-3-specific CD8⁺ T-cells exhibited a strong polarization to TNFα secretion as a solitary response (72%) with modest fractions secreting TNFα with degranulation (16%) or degranulation alone (10%) (FIGS. 3E white bars and 3G) with nearly no IFNγ secretion. By contrast, glypican-3-specific CD4+ T-cells a significantly broader array of response patterns with 45% manifesting more than 1 cytokine and/or degranulation function % (FIGS. 3E black bars and 3G). The polarization of CD8⁺ T-cell responses was antigen-specific and not global as CEF-expanded CD8⁺ T-cells exhibited a predominant IFNγ response (FIG. 3F) that was most often multicytokine and associated with degranulation.

Proliferation of Glypican-3-Specific CD8⁺ T-Cells In Vitro was not Associated with Restoration of Cytokine Responses in HCC Patients

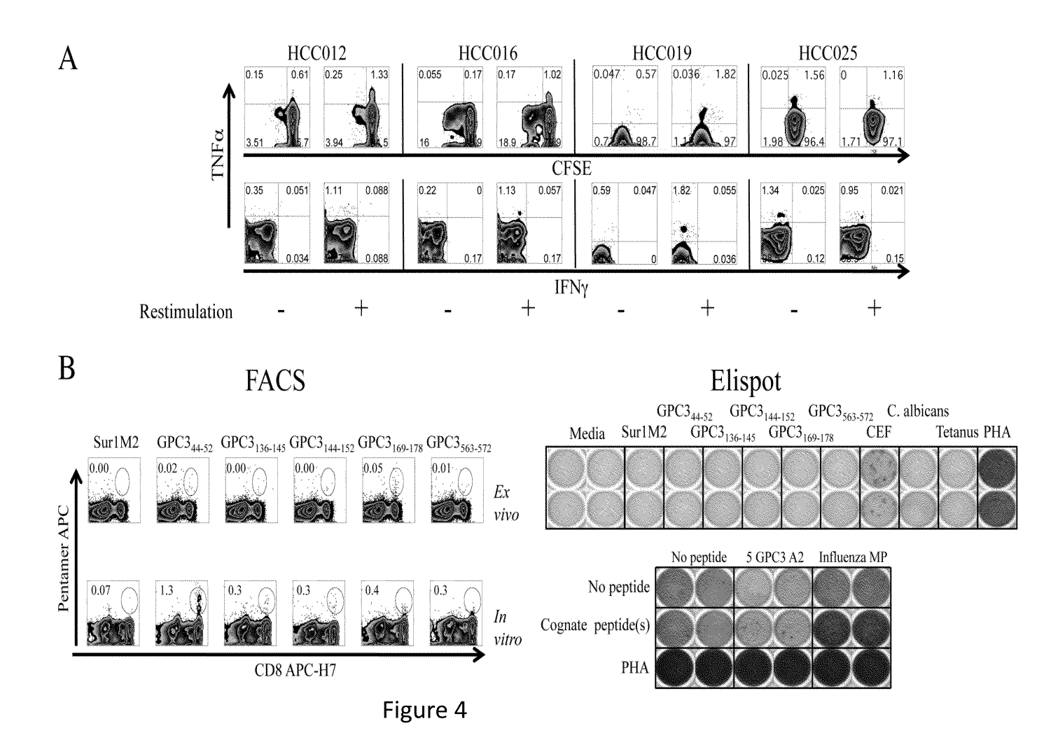

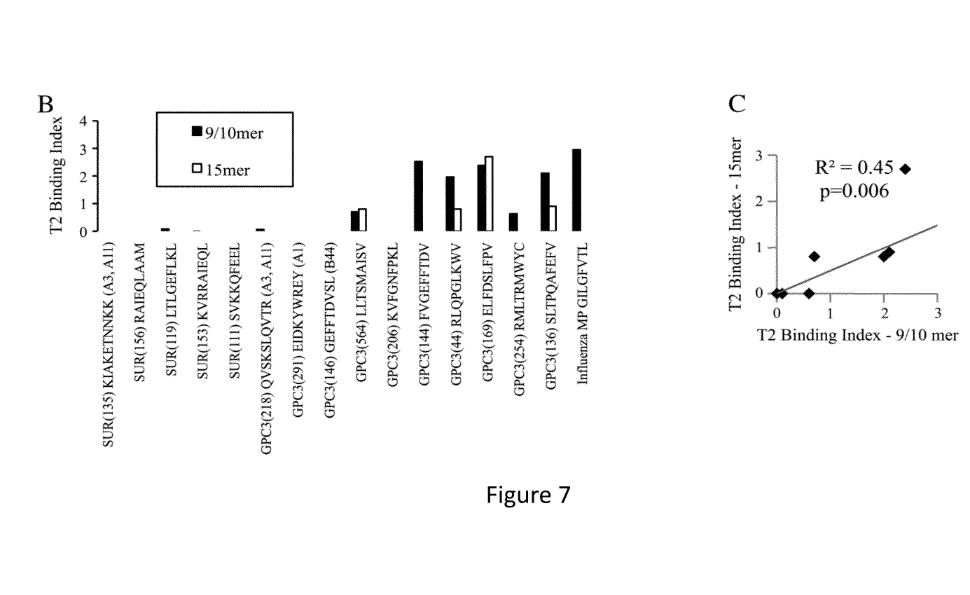

In a subset of patients in whom CFSE-labeling was utilized, as shown with 4 representative patients in FIG. 4A, glypican-3 peptide expansion in the presence of IL-2 led to modest T-cell proliferation (dilution of CFSE), but proliferating CD8+ T-cells did not develop the capacity to secrete cytokine or degranulate; by contrast, peptide-specific responses were found exclusively in mono-functional TNFα-producing non-proliferating cells. While these 15 mer peptides efficiently bind to class I MHC molecules (FIG. 7), to exclude the possibility that CD8⁺ T-cell polarization was solely attributable to the use of 15 mer rather than 9-10 mer optimal peptides, experiments were performed to use 9-10 mer optimal peptides to expand glypican-3-specific CD8+ T-cells in HLA-A2⁺ patients and tracked responder cell function by pentamer staining As shown in FIG. 4B for one representative patient, despite readily detectable expansion of GPC3₄₄₋₅₂ CD8+ T-cells after 10 days of in vitro expansion, IFNγ secretion by expanded cells restimulated with cognate antigens was minimal-to-absent, particularly when compared to influenza-specific controls. Furthermore, these glypican-3-specific CD8⁺ T-cells did not produce detectable amounts of IFNγ or TNFα by intracellular staining. In total, while modest expansion of glypican-3-specific CD8⁺ T-cells may occur with stimulation by 15 mer peptides in approximately half of HCC patients, despite the activation of CD4⁺ T-cell type 1 response, expanded CD8+ T-cells do not gain multifunctional effector capacity. CD8⁺ T-cell cytokine responses that are detected are produced by non-dividing CD8⁺ cells constrained to produce TNFα.

### PD-L1 and CTLA-4 Blockade do not Significantly Enhance the Expansion or Cytokine Production of Peripheral Glypican-3-Specific T-Cells

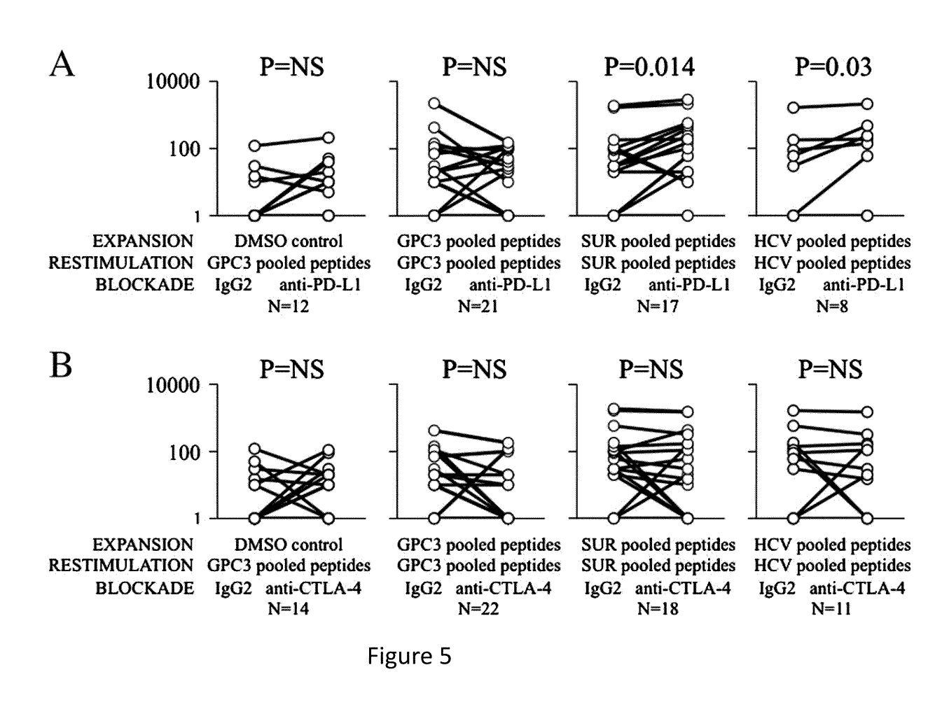

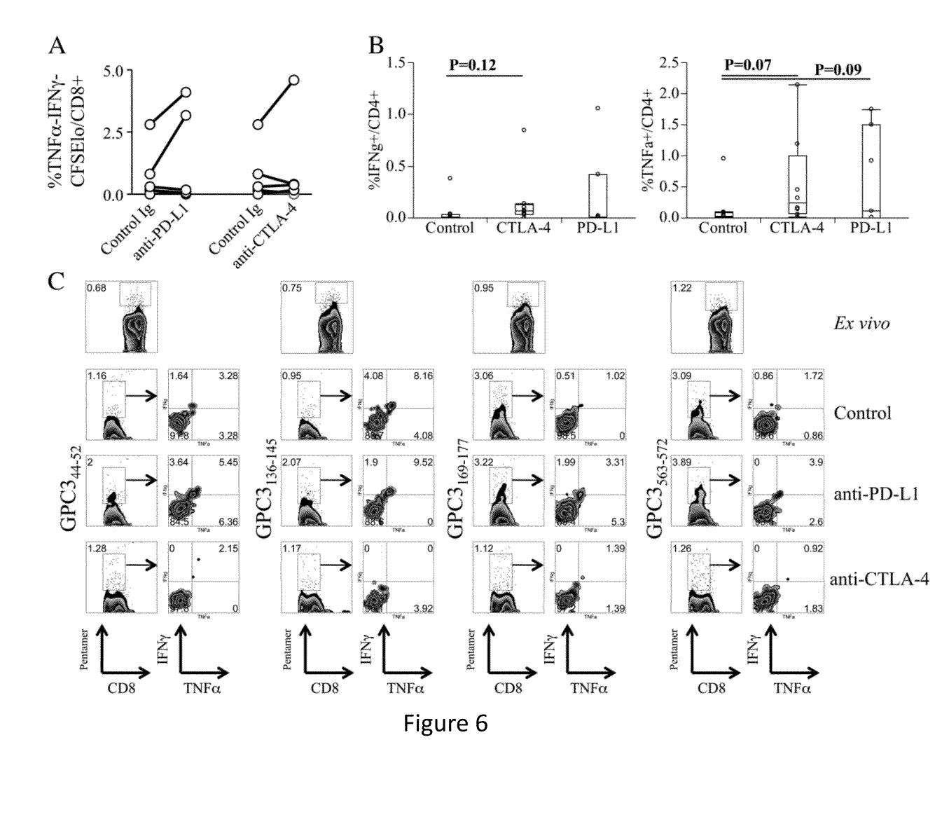

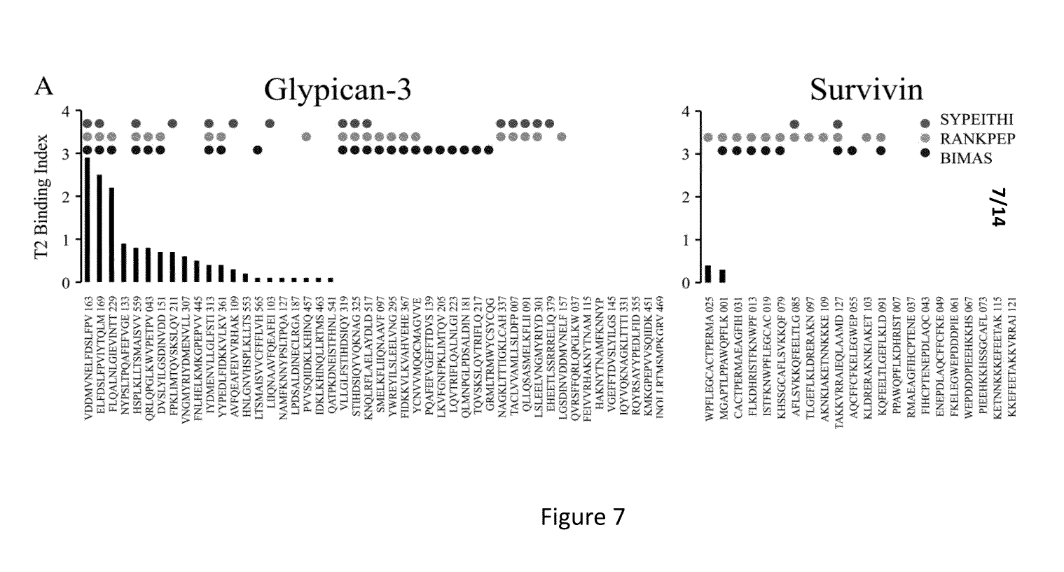

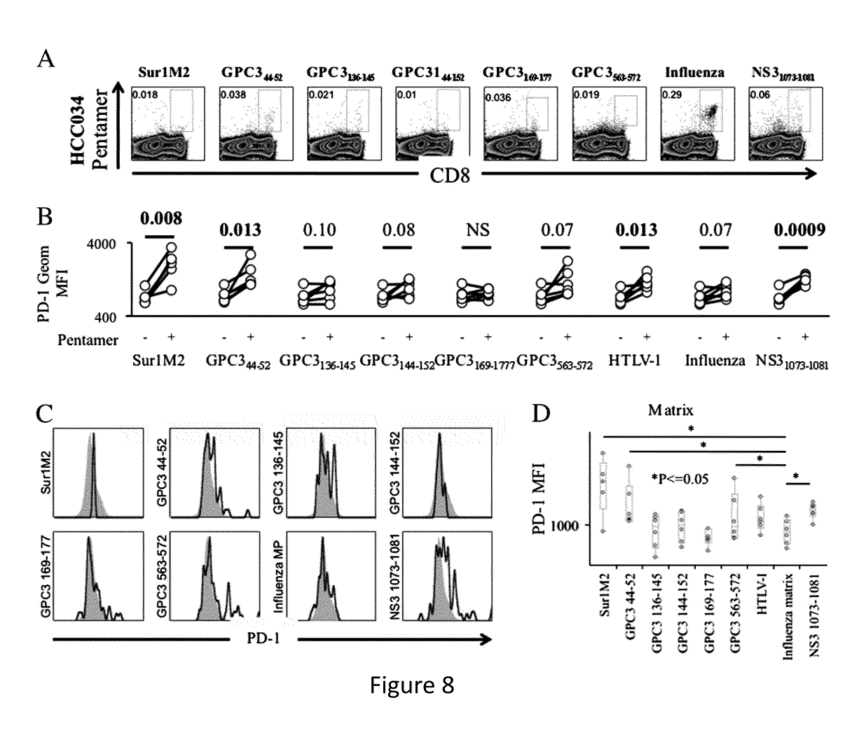

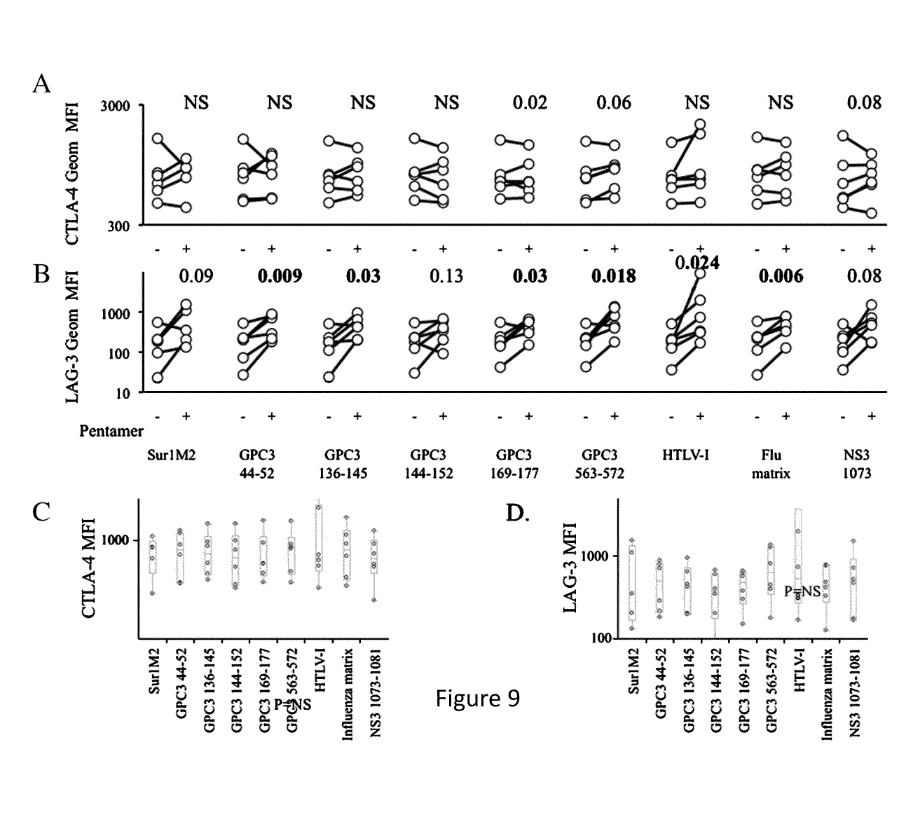

Inhibitory co-stimulation via the PD-1 and CTLA-4 receptors have been previously been shown to be important in suppressing hepatitis virus-specific T-cell effector functions, and PD-1 expression by tumor antigen-specific T-cells may be important in limiting T-cell reactivity to HCC in situ (Gao, et al., 2009, Clin Cancer Res 15:971-979; Shi, et al., 2011, Int J Cancer 128:887-896). Utilizing glypican-3-specific HLA-A2-restricted pentamers, the expression of PD-1 on peripheral glypican-3-specific CD8⁺ T-cells, which was detectable ex vivo in 7/9 HCC patients tested ranging in frequency from 0.03 to 1.6% of CD8⁺ T-cells was confirmed (Table 3). As shown in FIG. 8, PD-1 expression was significantly greater in pentamer-positive versus pentamer-negative CD8⁺ T-cells for the GPC3₄₄₋₅₂ (p=0.013) and GPC3₅₆₃₋₅₇₂ (strong trend p<0.07) epitopes. By contrast, CTLA-4 expression for GPC3-specific or control CD8+ T-cells was not strongly upregulated (FIG. 9). Based on these data, CFSE-labeled T-cells from HCC patients were expanded for 7 days in vitro in the presence glypican-3 peptides, rhIL-2, with either control Ig, anti-PD-L1 mAb, anti-CTLA-4 mAb, or combined PD-L1/CTLA-4 blockade to determine the effect of inhibitory co-stimulation blockade on glypican-3-specific T-cell responses. As shown in FIGS. 5A-5B, PD-L1 and CTLA-4 had no effect on glypican-3-specific IFNγ⁺ T-cell responses by Elispot. By contrast, surviving-specific and HCV NS3 responses were augmented by PD-L1. Dual blockade in 12 patients showed no suggestion of effect. With PD-L1 and CTLA-4 blockade, 2/7 and 1/7 patients respectively showed significant increases in CD8⁺ T-cell proliferation (FIG. 6A). However, when assessing mean peptide-induced proliferative and cytokine responses there was no significant difference in CD8⁺ T-cell proliferation, TNFα production, or IFNγ production under blockade conditions. By contrast, CTLA-4 blockade produced a trend towards a small median increase in CD4⁺ T-cell IFNγ and TNFα production while PD-L1 blockade also led to trend in CD4⁺ TNFα production (FIG. 6B). In one patient in which inhibitory receptor blockade could be combined with pentamer analysis, PD-L1 blockade doubled the frequency of GPC3₄₄₋₅₂- and GPC3₁₃₆₋₁₄₅-specific CD8⁺ T-cells after 7 days of expansion; however cytokine production appeared only minimally affected similar to findings with other HCC antigens (Gehring, et al., 2009, Gastroenterology 137:682-690). In aggregate, these data suggest that PD-1 and CTLA-4 blockade might modestly increase the proliferation of a subset of tumor antigen-specific T-cells in a minority of HCC patients but that co-stimulation blockade does not effectively restore effector function to peripheral tumor-specific CD8⁺ T-cells.

TABLE 3
Frequency of CD8⁺ pentamer T-cells in
nine HLA-Ar hepatocellular carcinoma patients.

| | No. of | No. with pentamer + | Median pentamer+/ |
|---|---|---|---|
| Pentamer | patients | cluster (%) | CD8+ cells (range) |
| 1 SurlM2 | 9 | 1 (11) | 0.20 (0.20-0.20) |
| 2 GPC3 44-52 | 9 | 3 (33) | 0.20 (0.10-0.30) |
| 3 GPC3 136-145 | 8 | 4 (50) | 0.70 (0.05-1.30) |
| 4 GPC3 142-151 | 9 | 2 (22) | 0.47 (0.03-0.90) |
| 5 GPC3 169-178 | 8 | 5 (63) | 1.00 (0.20-1.60) |
| 6 GPC3 563-572 | 9 | 4 (44) | 0.45 (0.05-1.60) |
| 7 HTLV-I | 7 | 1 (14) | 1.00 (0.10-1.00) |
| 8 Flu matrix | 9 | 5 (56) | 0.40 (0.30-1.30) |
| 9 NS3 1073 | 7 | 2 (29) | 0.07 (0.03-0.10) |

### Expansion of Interferon-Gamma-Producing T Cells

Among tumor antigens that have been considered as immunotherapeutic targets for hepatocellular carcinoma, glypican-3 has several attractive properties including its high tumor-to-background expression, relatively low extra-hepatic expression, and potential for serological detection. This study was performed to determine the frequency and functional capacity of circulating glypican-3-specific CD4⁺ and CD8⁺ T-cells in an HLA-diverse HCC population with underlying cirrhosis, the population most likely to be considered for a therapeutic vaccine. The results presented herein demonstrate that IFNγ+ T-cells specific for glypican-3 (as well as those specific for survivin, another tumor antigen relevant to HCC) were not detected from the population of cells cultured ex vivo using 15 mer overlapping peptide libraries and peptide matrices. Interestingly, a small minority of HCC patients did have T-cell responses against recombinant glypican-3, likely derived from CD4⁺ T-cells. Consistent with previous studies that utilized optimal HCC-related HLA-restricted epitopes in vitro (Komori, et al., 2006, Clin Cancer Res 12:2689-2697; Butterfield, et al., 2003, Clin. Cancer Res 9:5902-5908; Mizukoshi, et al., 2006, Hepatology 43:1284-1294; Zhang, et al., 2007, Cancer Immunol Immunother 56:1945-1954), expansion of glypican-3-specific T-cells from approximately half of HCC patients was successful, particularly in patients with evidence of higher serum antigen levels suggesting significant tumor-induced priming. Expansion using the 15 mer peptide library approach in an HLA-independent manner generated both CD4⁺ and CD8⁺ glypican-3-specific T-cells. Glypican-3-specific CD4⁺ T-cells retained or gained multiple effector functions during expansion. By contrast, expanded glypican-3-specific CD8⁺ T-cells failed to degranulate or to produce IFNγ when restimulated and were functionally limited to the production of TNFα. This CD8⁺ T-cell dysfunction could not be overcome by inhibition of PD-1 and/or CTLA-4 signaling. Thus, despite the presence of Th1-like CD4⁺ T-cell response, tumor-specific CD8+ T-cells in HCC patients manifest a deeply exhausted or anergized phenotype that could not be reversed simply with inhibitory co-stimulation blockade.

To date, clinical studies using class I peptide-based tumor vaccination for HCC have yet to show convincing, sustained clinical benefit (Butterfield, et al., 2003, Clin. Cancer Res 9:5902-5908; Butterfield, et al., 2006, Clin Cancer Res 12:2817-2825) while non-specific cytokine-activated lymphocytes (Lygidakis, et al., 1995, J. Interferon Cytokine Res 15:467-472; Weng, et al., 2008, J Immunother 31:63-71) and/or stimulation with cell lysate-loaded APCs (Palmer, et al., 2009, Hepatology 49:124-132) after resection or ablation of tumors has demonstrated some evidence of clinical efficacy. While the lack of benefit of peptide-based approaches could be due to selection of advanced patients unlikely to benefit from inclusion, the results presented herein, which are similar to the findings that utilizing NY-ESO-1b₁₅₇₋₁₆₅-specific CD8⁺ T-cells (Shang, et al., 2004, Clin Cancer Res 10:6946-6955) and panels of HLA-A2-restricted tumor antigens (Gehring, et al., 2009, Gastroenterology 137:682-690), suggest that peptide-expanded CD8⁺ T-cells often remain functionally impaired. Such dysfunction could be due to aberrant liver-specific priming of CD8⁺ T-cells (Su, et al., 2010, J. Immunol. 185:7498-7506) or tumor-induced upregulation of multiple inhibitory receptors on these T-cells to inhibit TCR-induced activation (Vazquez-Cintron, et al., 2010, J Immunol 185:7133-7140).

Blockade of inhibitory co-stimulation pathways is an emerging approach to overcome tumor-specific CD8⁺ T-cell dysfunction for the treatment of solid tumors (Brahmer, et al., 2010, J Clin Oncol 28:3167-3175; Royal, et al., 2010, J Immunother 33:828-833). The results presented herein strongly implicate the role of inhibitory co-stimulation by PD-1 and CTLA-4 in the suppression of CD8⁺ T-cells in chronic hepatitis C (Nakamoto, et al., 2008 Gastroenterology 134(7):1927-37), the underlying etiology for HCC in 32/33 patients we studied. The expression of PD-L1, an inducible ligand for PD-1, has been to shown to impart a poor prognosis in HCC (Gao, et al., 2009, Clin Cancer Res 15:971-979). PD-L1 is selectively upregulated on malignant hepatocytes and/or tumor-associated Kupffer cells as a result of T-cell-derived IFNγ or tumor-related IL-10, and reversibly inhibits effector PD-1⁺ CD8⁺ T-cell proliferation in some studies (Shi, et al., 2011, Int J Cancer 128:887-896) and (Wu, et al., 2009, Cancer Res 69:8067-8075). By contrast, the results presented herein indicate that only modest expression of PD-1 on tumor antigen-specific peripheral blood CD8⁺ T-cells in HCC patients was detected, possibly reflecting compartmentalization of PD-1^{hi} activated T-cells to the target environment, a finding suggested by other investigators using different HCC-associated tumor antigen-derived epitopes (Gehring, et al., 2009, Gastroenterology 137:682-690). While peripheral CD8⁺ T-cells specific for a subset of glypican-3 epitopes do express elevated levels of PD-1 (but not CTLA-4 and not specifically LAG-3), functional rescue in vitro by PD-L1 blockade did not markedly improve T-cell proliferation, cytokine production or degranulation both in assays using specific HLA-A2 epitopes and the 15 mer peptide library. These data contrast with HCV-specific responses which in this and previous work (Nakamoto, et al., 2008 Gastroenterology 134(7):1927-37) were augmented by PD-L1 blockade. While survivin-specific T-cell IFNγ responses were modestly increased by PD-L1 blockade, overall these data suggest that inhibitory co-stimulation blockade is unlikely to potently augment peripheral T-cell vaccination responses in HCC. However, studies on PBMC cannot be assumed to wholly reflect the impact of interventions within a tumor. Indeed, recent data suggest that PD-L1 blockade in vivo may stimulate effector T-cells in an indirect manner via reduction of the generation of intratumoral Tregs (Zhou, et al., 2010, J Immunol 185:5082-5092). Thus, the results presented herein support the investigation of both direct and indirect effects of PD-1 blockade when such therapies are tested in patients.

The expansion and characterization of glypican-3-specific CD4⁺ T-cells presented herein is a novel finding in that, along with data relating to other tumor antigen-specific CD4⁺ T-cells in HCC, further highlights a population of cells that might be an important target for immune augmentation. The potential role for CD4⁺ T-cells to exert a cytolytic effect on HCC has been suggested in both human and murine studies (Nakao, et al., 1997, Cell Immunol 177:176-181; Homma, et al., 2005, Immunology 115:451-461). The ability to detect CD4⁺ T-cells specific for NY-ESO-1 ex vivo has been described in a small number of HCC patients in one study (Korangy, et al., 2004, Clin Cancer Res 10:4332-4341). The detection of AFP-specific CD4+ T-cells has generally required short-term in vitro expansion (Alisa, et al., 2005, Clin Cancer Res 11:6686-6694; Witkowski, et al., 2011 Int J. Cancer. 129, 2171-2182; Behboudi, et al., 2010, Br J Cancer 102:748-753), resulting in the expansion of either effector (Alisa, et al., 2005, Clin Cancer Res 11:6686-6694; Behboudi, et al., 2010, Br J Cancer 102:748-753) or TGFβ+ regulatory T-cells (Alisa, et al., 2008, J Immunol 180:5109-5117). We were able to detect responses to recombinant glypican-3 by Elispot ex vivo in a small number of HCC patients, but no early fibrosis or cirrhotic patients, suggesting tumor-induced priming. Half of normal donors in our study had detectable ex vivo CD4⁺ T-cell responses yet these did not translate into in vitro expansion of glypican-3-specific CD4⁺ T-cells, also supporting the concept that tumor-priming is critical for establishing an expandable population of CD4+ T-cells (Alisa, et al., 2005, Clin Cancer Res 11:6686-6694). Further support for this concept comes from our finding that the presence of serum antigen correlated strongly with the expansion of glypican-3-specific CD4⁺ T-cells, as has been suggested by some (Witkowski, et al., 2011 Int J. Cancer. 129, 2171-2182) but not all (Behboudi, et al., 2010, Br J Cancer 102:748-753) studies with AFP. The recent study from Witkowski et al. (Witkowski, et al., 2011 Int J Cancer. 129, 2171-2182) demonstrates that the infrequent detection of HCC-reactive CD4⁺ T-cells ex vivo is not likely the result of compartmentalization of these cells into the tumors, nor was it likely due to circulation of dysfunctional CD4⁺ T-cells, but rather due to low circulating precursor frequencies of potential effector cells. While we did not specifically assess TGFβ secretion (Alisa, et al., 2008, J Immunol 180:5109-5117) or a regulatory phenotype in expanded glypican-3-specific CD4⁺ T-cells, it is unlikely that the peripheral CD4⁺ T-cells were regulatory given the Tₕ1-like functional profiles. In unpublished observations, we have found that roughly one-third of intratumoral CD4⁺ T-cells expressed foxp3 and CD25, and that TIL can suppress of glypican-3-specific IFNγ production among TIL and LIL possibly mediated by antigen-specific IL-10 suggesting possible antigen-specific Treg accumulation within the tumors. While the expansion of tumor-reactive peripheral CD4⁺ T-cells in tumor-bearing patients might support the use of whole-antigen CD4⁺ T-cell-directed components for therapeutic vaccination, the lack of generation of multifunctional CD8⁺ T-cells despite the presence of significant CD4⁺ T-cell responses in our system argues that CD4-directed approaches to increase “help” may not sufficiently help highly dysfunction CD8⁺ T-cells to regain multi-effector function.

The results presented herein demonstrate that 15 mer peptide libraries can expand CD4⁺ and CD8⁺ T-cells specific for glypican-3 and survivin in patients with hepatocellular carcinoma in an HLA-nonspecific manner. The CD4⁺ T-cells retained a broad array of cytokine-secreting capacity, but glypican-3-reactive CD8⁺ T-cells were functionally constrained suggesting a differentiated or exhausted state. However, this exhausted state could not be reversed by inhibitory co-stimulation blockade in vitro unlike the exhausted state of virus-specific T-cells in chronic viral hepatitis. Without wishing to be bound by any particular theory, whole protein or peptide library-based stimulation of tumor antigen-specific T-cells may be able to generate effector CD4⁺ T-cell responses in a large proportion of hepatocellular carcinoma patients. In addition, further studies can be designed to further define optimal conditions, including the need for concomitant ablation of the tumor microenvironment, for the concomitant expansion of multifunctional effector CD8+ T-cells.

### Example 2

### Expansion of CD8+ T-Cells Isolated from Normal Donors and Hepatocellular Carcinoma Patients Using Monocyte-Derived Dendritic Cells and Defined HLA-A2-Restricted Antigens

Experiments were designed to demonstrate that tumor antigen-specific CD8⁺ T-cells expanded from HLA-A2⁺ healthy donors using optimized antigen-presenting cells possess significantly greater affinity for their target antigen than similarly generated CD8⁺ T-cells from HLA-A2⁺ patients with hepatocellular carcinoma. In addition, experiments were designed to demonstrate that expression of cloned high-affinity HLA-A2-restricted tumor antigen-specific T-cell receptors (TCRs) derived from healthy donors in polyclonal T-cells from cirrhotic HCC patients confer potent tumor antigen-specific effector functions.

Briefly, CD8+ T-cells isolated from normal donors and hepatocellular carcinoma patients are expanded using monocyte-derived dendritic cells and defined HLA-A2-restricted antigens. The affinity and effector function of expanded T-cells are determined using approaches described elsewhere herein. High-affinity T-cell clones can be isolated using HLA-A2 pentamers by flow cytometry. Whole cellular DNA can be isolated and cloned. For example, the T-cell receptor alpha and beta mRNA from these isolated T-cells can be amplified and cloned for further analysis.

The cloned alpha and beta TCR subunits can be transduced into T-cells from cirrhotic liver cancer patients to confirm that transfer of a high-affinity TCR can restore antigen-specific effector function to these cells. Once validated in vitro, these TCR can then be developed into a therapeutic modality first in vivo in animal models and then, if effective and safe, in human studies. In support of the approach for these aims, several lines of preliminary work discussed elsewhere herein have been completed which demonstrate the feasibility of the proposed strategy.

The materials and methods employed in these experiments are now described.

A Cohort of HLA-A2⁺ HCC Patients and Normal Donors

Under a current IRB-approved protocol (Cellular Immunity in Hepatocellular Carcinoma #00988), patients with HCC, cirrhosis, non-cirrhotic hepatitis and healthy controls from the Philadelphia VA Medical Center have been recruited. The cohort includes 37 HCC patients, 15 cirrhotic non-tumor controls and 21 non-tumor non-cirrhotic controls. 11/37 HCC and 7/15 normal donors are HLA-A2⁺. Frozen PBMC are available for the majority of the HCC patients and all normal donors and are ready for immediate utilization.

Glypican-3-Specific Reagents Including HLA-A2 Pentamers

A 15 mer overlapping peptide library of glypican-3 peptides (protein length 580aa, 95 peptides) was synthesized commercially (Proimmune, Oxford UK). Optimum HLA-A2-binding epitopes were designed initially by using online algorithms then validated using a T2 binding assay. MHC Class I HLA-A2.1-restricted pentamers were synthesized for the following GPC3 epitopes that were found to have strong binding affinity: GPC3₄₄₋₅₂ (RLQPGLKWV; SEQ ID NO: 2), GPC3₁₃₆₋₁₄₅ (SLTPQAFEFV; SEQ ID NO: 3), GPC3₁₄₂₋₁₅₁ (FEFVGEFFTD; SEQ ID NO: 4), GPC3₁₆₉₋₁₇₇ (ELFDSLFPV; SEQ ID NO: 5), and GPC3₅₆₃₋₅₇₂ (KLLTSMAISV; SEQ ID NO: 6). Controls included Sur1M2, a previously validated survivin-specific epitope, influenza matrix and HCV NS3 1073-1081 virus-specific pentamers. Each pentamer structure consists of a central fluorochrome and five attached epitope-expressing HLA monomers that allows for flow cytometric detection and isolation of epitope-specific CD8⁺ T-cells.

The results of the experiments are now described.

Glypican-3-Specific CD8⁺ T-Cells can be Detected from Peripheral Blood of HLA-A2⁺ HCC Patients and from Normal Donors

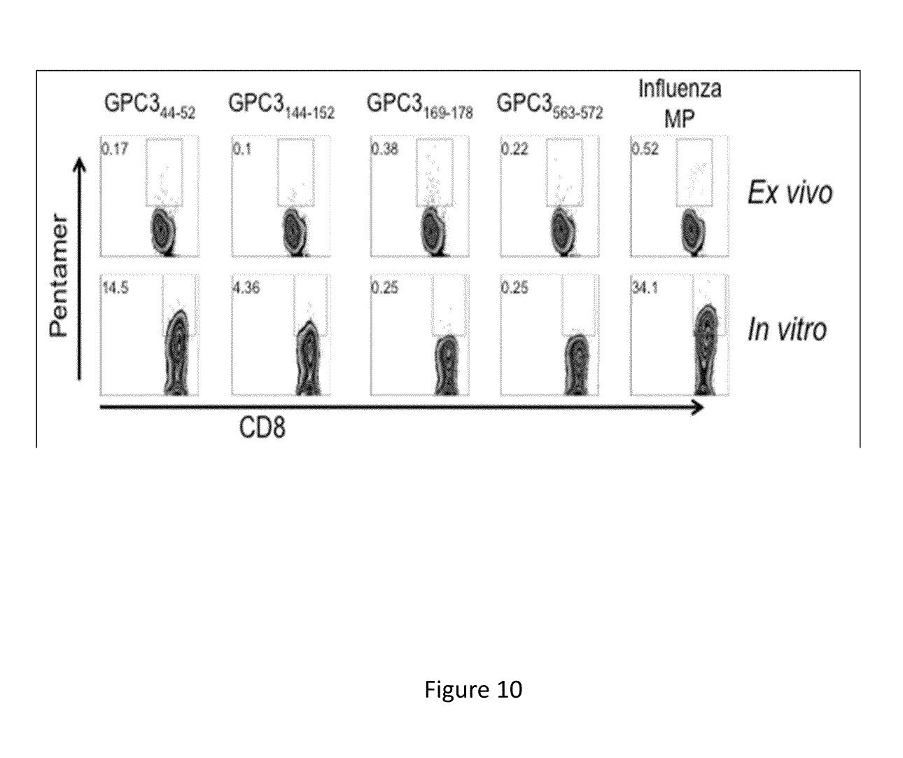

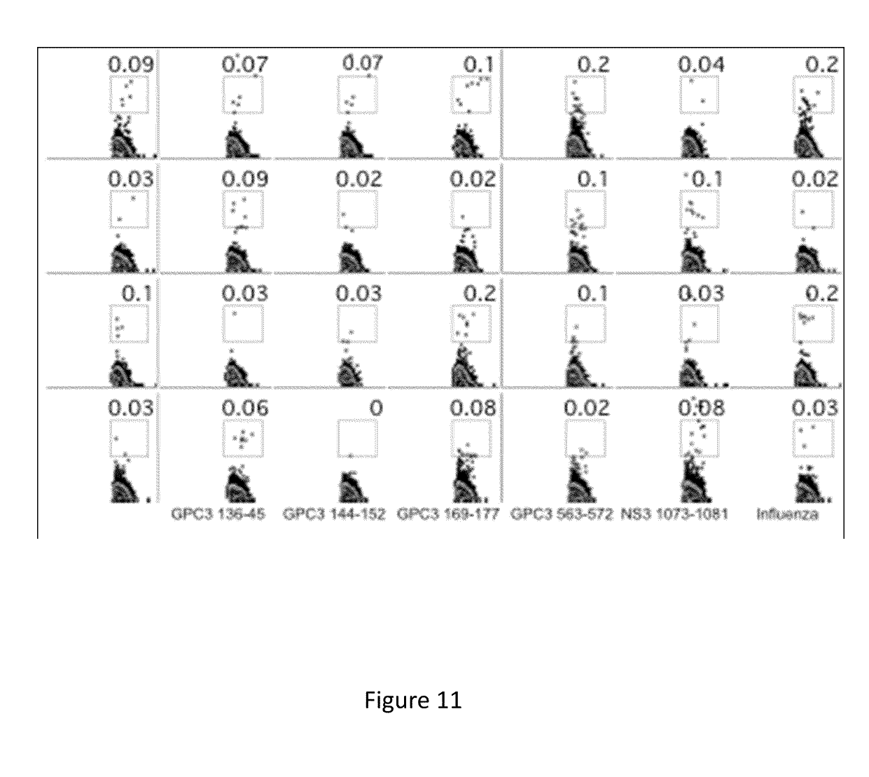

The five HLA-A2-restricted pentamers were used to detect CD8⁺ T-cells circulating in the peripheral blood of patients with HCC and normal donors. FIG. 10 demonstrates the isolation and expansion of circulating GPC3-specific CD8⁺ T-cells from one of three HLA-A2⁺ HCC patients. While low levels of pentamer-binding CD8⁺ T-cells could be detected ex vivo, a significant expansion of epitope-specific CD8⁺ T-cells could be detected after stimulation of PBMC for one week in vitro in the presence of all 5 glypican-3 optimal peptides and rhIL-2. Influenza-specific CD8⁺ T-cells were also markedly expanded under similar conditions. The fluorescence intensity of pentamer-binding cells appeared relatively lower than influenza-specific cells, suggesting a lower affinity interaction. Additional studies ex vivo in HLA-A2⁺ HCC patients show that these patients harbor GPC3-specific CD8⁺ T-cells at a precursor frequency of ˜0.2-0.5%. Studies of 4 HLA-A2⁺ subjects without liver disease demonstrate that clusters of glypican-3-specific CD8⁺ T-cells can be detected at low frequency even in normal subjects (FIG. 11). Thus, the data presented herein suggest that similar to other malignancies, HCC antigen-specific T-cells can be detected using sensitive techniques in patients who do not have hepatocellular carcinoma.

Tumor Antigen-Specific T-Cells can be Detected after Short-Term In Vitro Expansion from Peripheral Blood from Both Cancer Patients and Normal Donors

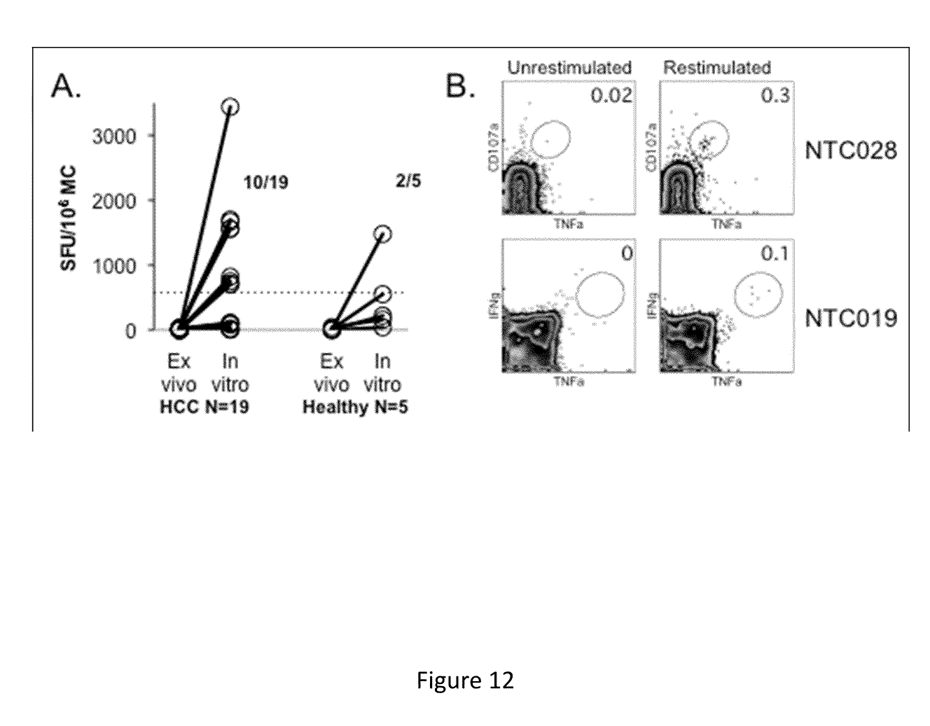

Given the low expected and observed frequency of effector T-cell responses against tumor antigens in patients, T-cell responses against glypican-3 and another tumor-related antigen, survivin, before and after short-term in vitro expansion using 15 mer overlapping peptides were further examined. In an IFNγ Elispot assay, while neither HCC patients nor normal donors had IFNγ responses ex vivo, 10/19 (52%) HCC patients and 2/5 (40%) normal donors had detectable responses against glypican-3 after one week of in vitro expansion (FIG. 12). The dominant responder cell population in HCC patients was CD4⁺ T-cells, and CD8⁺ T-cells that expanded weakly exclusively produced TNFα, did not produce IFNγ, and did not degranulate. By contrast, CD8⁺ T-cells detected after expansion in healthy donors produced multiple cytokines and/or evidence of degranulation when restimulated (NTC028 and NTC019 shown in right side of FIG. 12). Thus, in normal donors, glypican-3-specific CD8⁺ T-cells with potential multifunctional effector capacity circulate at low but detectable precursor frequency and are expandable in a significant fraction of healthy individuals. These data are important for the feasibility of generating tumor antigen-specific CD8⁺ T-cells expanded from HLA-A2⁺ healthy donors using optimized antigen-presenting cells to possess significantly greater affinity for their target antigen than similarly generated CD8⁺ T-cells from HLA-A2⁺ patients with hepatocellular carcinoma.

### Example 3

### Use of Glypican-3 Peptide-Pulsed Autologous Monocyte-Derived Dendritic Cells

Using glypican-3 peptide-pulsed autologous monocyte-derived dendritic cells, glypican-3-specific CD8⁺ T-cells from 5 HLA-A2⁺ normal donors and 5 HLA-A2⁺ HCC patients can be expanded. In some instances, the cells are re-stimulated weekly for a total of 4 cycles in the presence of rhIL-2. After 28 days, expanded CD8⁺ T-cells can be tested for HLA-A2-pentamer binding, epitope-specific cytolysis, and MHC-TCR binding avidity via pentamer binding decay studies.

The materials and methods employed in these experiments are now described.

Samples from Patients

Cryopreserved and/or fresh PBMC from 5 normal donor and 5 HCC HLA-A2⁺ patients will be utilized under an existing active IRB protocol (#00988 Cellular Immunity in Hepatocellular Carcinoma). This protocol allows collection of up to 1 unit (˜450 ml) of peripheral blood over a 3-month period from donors; however, for the proposed experiments 50 ml blood per subject may be sufficient. After thawing cryopreserved PBMC or density centrifugation of fresh lymphocytes, CD8⁺ T-cells are negatively selected using magnetic bead separation kits (Miltenyi Biotec) prior to expansion.

48 h Dendritic Cell Preparation

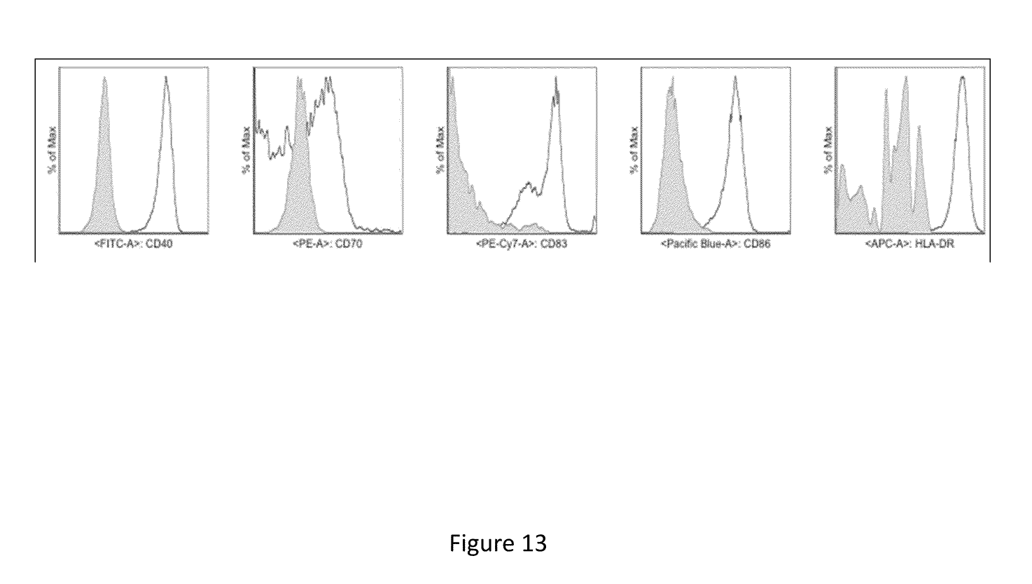

Monocytes are separated from PBMC using adherence to cell culture plastics using standard procedures. The cells are then supplemented with GM-CSF and IL-4. After 24 hours of incubation, the cells are supplemented with a cocktail of maturation cytokines (GM-CSF, IL-4, TNFα, IL-1β, IL-6 and PGE₂). DC development under these conditions have been piloted in the laboratory with significant upregulation of MHC I, MHC 11, CD83 and CD86 (FIG. 13). DCs are then harvested after 24 hours and primed with the GPC3₄₄₋₅₂ or GPC3₁₆₉₋₁₇₇ peptides at either low-concentration (0.2 uM—to expand high affinity T-cells) or high concentration (20 uM—to expand high or low affinity T-cells) and then irradiated prior to incubating with freshly isolated CD8⁺ T-cells. Controls for CD8⁺ T-cell expansion include CMVpp65, Alphafetoprotein (AFP)₁₅₈₋₁₆₆ and influenza matrix peptide. AFP₁₅₈₋₁₆₆ can be used as a test condition due to the widespread use of AFP as a tumor marker for HCC.

CD8⁺ T-Cell Expansion

CD8⁺ cells can be co-cultured with the primed DC cell preparation at a ratio of 5:1 CTLs:DC in complete medium. Starting on day 3 and then every 3-4 days, cultures are supplemented with rhIL-7 and rhIL-2. On day 7, 14 and 21, CD8⁺ T-cells are re-stimulated by peptide-pulsed irradiated autologous PBMC at either low—(0.2 uM) or high (20 uM) peptide concentration for the specific GPC3₄₄₋₅₂ or GPC3₁₆₉₋₁₇₇ peptide or the control peptides.

Pentamer Intracellular Cytokine Staining

At day 27-28 of expansion, T-cell lines are harvested and counted. An unstimulated sample can be stained with the cognate and non-cognate pentamers to confirm specificity. 2×10⁵ cells will then be co-cultured with mock-pulsed T2 cells (an HLA-A2⁺ TAP-deficient cell line that can only present exogenous peptides), cognate peptide-pulsed T2 cells, and PMA/ionomycin for 6 hours in the presence of anti-CD107a antibody (a degranulation probe) and monensin, then stained intracellularly for IFNγ and TNFα.

Cytotoxicity Assessment

Standard ⁵¹Cr-release assays using cognate peptide-loaded T2 and CD8⁺ T-cell clones at increasing E:T ratios can be used to identify the LD₅₀ of each expanded T-cell clone. Briefly, T2 cells are loaded with 1 uM of cognate GPC3 epitope (or control CMV and Flu), labeled with 100 μCi Na₂[⁵¹Cr]O₄, then co-cultured for 4 hours with CD8⁺ T-cell clones at increasing E:T ratios. Percentage specific lysis can be calculated in a standard manner. Cytotoxicity and HLA-restriction can be confirmed in HepG2 (HLA-A2⁺, glypican-3-expressing) and Hep3B (HLA-A2⁻, glypican-3-expressing) hepatoma cells.

### Example 4

### TCR Clones

GPC3₄₄₋₅₂- or GPC3₁₆₉₋₁₇₇-specific T-cell clones can be used to isolate desired TCR clones. A high affinity alphafetoprotein-specific TCR (targeting AFP₁₅₈₋₁₆₆) can also be used to demonstrate that expression of cloned high-affinity HLA-A2-restricted tumor antigen-specific T-cell receptors (TCRs) derived from healthy donors in polyclonal T-cells from cirrhotic HCC patients confer potent tumor antigen-specific effector functions.

RNA for each TCR can be introduced into naïve CD8⁺ T-cells from patients with HCC. The transduced CD8⁺ T-cells' peptide-specific effector functions of cytokine expression and cytolytic activity is accessed.

The materials and methods employed in these experiments are now described.

Isolation of Antigen-Specific CD8⁺ T-Cells

Based on binding affinity assays discussed elsewhere herein, at least two (2) T-cell lines can be chosen for further study—one high and one low affinity against either GPC3₄₄₋₅₂ or GPC3₁₆₉₋₁₇₇. Pentamer-specific T-cells can be isolated and purified using flow cytometric sorting and/or MACS-based enrichment. Final anticipated purity is at >99%.

TCR Cloning

We plan to clone the TCRα and β chains using a standard protocol but have also established collaboration with Dr. Bent Jakobsen at Immunocore Ltd. with expertise in TCR cloning and codon optimizination (see attached letter of support) for technical assistance. Briefly, total RNA from 5×10⁶ CTLs will be isolated using commercially available RNA extraction kits (Qiagen). cDNA will be reverse transcribed using both 64T-primer (which binds poly-A tail) and a capswitch oligonucleotide (which allows hybridization with a T7 primer). Initial amplification of cDNA will then be followed by further PCR amplification using α-chain or β-chain-specific 5′ and 3′ primers with confirmatory sequencing. Amplified TCR chains will then be cloned into an RNA-production vector pCR2.1 (Invitrogen).

Preparation of RNA for Transfer into Non-Specific T-Cells

Gene-specific 5′ primers include the T7 polymerase binding sequence, Kozak sequence, a start codon and the next 19-15 bp of Vα or Vβ region for each TCR. 3′ primers include 64T primer and 18-25 bp of the relevant Cα or Cβ sequence. PCR products can be gel purified, re-amplified, cleaned and used as templates for in vitro transcription using Ambion T7 mMESSAGE MACHINE for transient expression or using pELNS lentivirus for stable transfection.

Transient Expression of TCRα and β into Non-Specific T-Cells

CD8⁺ T-cells from HCC patients can be purified via negative selection and stimulated with anti-CD3/CD28 beads in the presence of rhIL-2. RNA for each TCR gene (0.5-2.0 μg of RNA per 10⁶ T-cell) can be electroporated. Electroporated cells are then rested for 16 hours before effector assays.

In-Vitro Assay for Cloned TCR Activation in the Setting of Exposure to GPC3

RNA electroporations can be repeated using CD8⁺ T-cells from three HCC donors. Transduced CD8⁺ T-cells can be co-cultured with cognate peptide-pulsed and mock-pulsed T2 cells at an E:T ratio of 1:1 for 6 hours in the presence of anti-CD107a mAb and monensin then intracellularly stained for IFNγ, IL-17A and TNFα. Cytolytic capacity can be assessed using co-cultures of ⁵¹Cr-loaded peptide-pulsed and mock-pulsed T2-cells at descending E:T ratios.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of expanding an antigen specific T cell in a population of cells, the method comprising isolating the population of cells from a human, contacting the population of cells with an MHC restricted antigenic glypican-3 peptide, thereby expanding an antigen specific T cell from the population of cells.

2. The method of claim 1, wherein the human is an HLA-A2⁺ healthy donor.

3. The method of claim 1, wherein the T cell is specific for glypican-3.

4. The method of claim 1, wherein the MHC restricted antigenic glypican-3 peptide comprises the sequence selected from the group consisting of SEQ ID NO: 2, SEQ ID NO: 3, SEQ ID NO: 4, SEQ ID NO: 5, and SEQ ID NO: 6.

5. The method of claim 1, wherein the population of cells comprises peripheral blood mononuclear cells (PBMCs).

6. An isolated antigen specific T cell generated by isolating a population of cells from a human, contacting the population of cells with an MHC restricted antigenic glypican-3 peptide, expanding an antigen specific T cell that is reactive to the glypican-3 peptide, and isolating the expanded antigen specific T cell using a multimer wherein the multimer comprises the glypican-3 peptide.

7. The cell of claim 6, wherein the human is an HLA-A2⁺ healthy donor.

8. The cell of claim 6, wherein the T cell is specific for glypican-3.

9. The cell claim 6, wherein the MHC restricted antigenic glypican-3 peptide comprises the sequence selected from the group consisting of SEQ ID NO: 2, SEQ ID NO: 3, SEQ ID NO: 4, SEQ ID NO: 5, and SEQ ID NO: 6.

10. The cell of claim 6, wherein the population of cells comprises peripheral blood mononuclear cells (PBMCs).

11. An isolated polynucleotide encoding a T cell receptor (TCR) that is derived from an antigen specific T cell, wherein the antigen specific T cell is produced by isolating a population of cells from a human, contacting the population of cells with an MHC restricted antigenic glypican-3 peptide, expanding an antigen specific T cell that is reactive to the glypican-3 peptide, and isolating the expanded antigen specific T cell using a pentamer wherein the pentamer comprises the glypican-3 peptide.

12. The cell of claim 11, wherein the human is an HLA-A2⁺ healthy donor.

13. The cell of claim 11, wherein the T cell is specific for glypican-3.

14. The cell claim 11, wherein the MHC restricted antigenic glypican-3 peptide comprises the sequence selected from the group consisting of SEQ ID NO: 2, SEQ ID NO: 3, SEQ ID NO: 4, SEQ ID NO: 5, and SEQ ID NO: 6.

15. The cell of claim 11, wherein the population of cells comprises peripheral blood mononuclear cells (PBMCs).


SEQUENCE LISTING (95 sequences)

SEQ ID NO: 1 - protein, 9 aa, Artificial sequence
  Chemically synthesized
LMLGEFLKL

SEQ ID NO: 2 - protein, 9 aa, Artificial sequence
  Chemically synthesized
RLQPGLKWV

SEQ ID NO: 3 - protein, 10 aa, Artificial sequence
  Chemically synthesized
SLTPQAFEFV

SEQ ID NO: 4 - protein, 10 aa, Artificial sequence
  Chemically synthesized
FEFVGEFFTD

SEQ ID NO: 5 - protein, 9 aa, Artificial sequence
  Chemically synthesized
ELFDSLFPV

SEQ ID NO: 6 - protein, 10 aa, Artificial sequence
  Chemically synthesized
KLLTSMAISV

SEQ ID NO: 7 - protein, 9 aa, Artificial sequence
  Chemically synthesized
LLFGYPVYV

SEQ ID NO: 8 - protein, 9 aa, Artificial sequence
  Chemically synthesized
CINGVCWTV

SEQ ID NO: 9 - protein, 9 aa, Artificial sequence
  Chemically synthesized
GILGFTFVL

SEQ ID NO: 10 - protein, 15 aa, Artificial sequence
  Chemically synthesized
VDDMVNELFDSLFPV

SEQ ID NO: 11 - protein, 15 aa, Artificial sequence
  Chemically synthesized
ELFDSLFPVIYTQLM

SEQ ID NO: 12 - protein, 15 aa, Artificial sequence
  Chemically synthesized
FLQALNLGIEVINTT

SEQ ID NO: 13 - protein, 15 aa, Artificial sequence
  Chemically synthesized
NYPSLTPQAFEFVGE

SEQ ID NO: 14 - protein, 15 aa, Artificial sequence
  Chemically synthesized
HSPLKLLTSMAISVV

SEQ ID NO: 15 - protein, 15 aa, Artificial sequence
  Chemically synthesized
QRLQPGLKWVPETPV

SEQ ID NO: 16 - protein, 15 aa, Artificial sequence
  Chemically synthesized
DVSLYILGSDINVDD

SEQ ID NO: 17 - protein, 15 aa, Artificial sequence
  Chemically synthesized
FPKLIMTQVSKSLQV

SEQ ID NO: 18 - protein, 15 aa, Artificial sequence
  Chemically synthesized
VNGMYRIYDMENVLL

SEQ ID NO: 19 - protein, 15 aa, Artificial sequence
  Chemically synthesized
FNLHELKMKGPEPVV

SEQ ID NO: 20 - protein, 15 aa, Artificial sequence
  Chemically synthesized
IYDMENVLLGLFSTI

SEQ ID NO: 21 - protein, 15 aa, Artificial sequence
  Chemically synthesized
YYPEDLFIDKKVLKV

SEQ ID NO: 22 - protein, 15 aa, Artificial sequence
  Chemically synthesized
AVFQEAFEIVVRHAK

SEQ ID NO: 23 - protein, 15 aa, Artificial sequence
  Chemically synthesized
HNLGNVHSPLKLLTS

SEQ ID NO: 24 - protein, 16 aa, Artificial sequence
  Chemically synthesized
LTSMAISVVCFFFLVH

SEQ ID NO: 25 - protein, 15 aa, Artificial sequence
  Chemically synthesized
LIIQNAAVFQEAFEI

SEQ ID NO: 26 - protein, 15 aa, Artificial sequence
  Chemically synthesized
NAMFKNNYPSLTPQA

SEQ ID NO: 27 - protein, 15 aa, Artificial sequence
  Chemically synthesized
LPDSALDINECLRGA

SEQ ID NO: 28 - protein, 15 aa, Artificial sequence
  Chemically synthesized
PVVSQIIDKLKHINQ

SEQ ID NO: 29 - protein, 15 aa, Artificial sequence
  Chemically synthesized
IDKLKHINQLLRTMS

SEQ ID NO: 30 - protein, 15 aa, Artificial sequence
  Chemically synthesized
QATPKDNEISTFHNL

SEQ ID NO: 31 - protein, 15 aa, Artificial sequence
  Chemically synthesized
VLLGLFSTIHDSIQY

SEQ ID NO: 32 - protein, 15 aa, Artificial sequence
  Chemically synthesized
STIHDSIQYVQKNAG

SEQ ID NO: 33 - protein, 15 aa, Artificial sequence
  Chemically synthesized
KNQLRFLAELAYDLD

SEQ ID NO: 34 - protein, 15 aa, Artificial sequence
  Chemically synthesized
SMELKFLIIQNAAVF

SEQ ID NO: 35 - protein, 15 aa, Artificial sequence
  Chemically synthesized
YWREYILSLEELVNG

SEQ ID NO: 36 - protein, 15 aa, Artificial sequence
  Chemically synthesized
FIDKKVLKVAHVEHE

SEQ ID NO: 37 - protein, 15 aa, Artificial sequence
  Chemically synthesized
YCNVVMQGCMAGVVE

SEQ ID NO: 38 - protein, 15 aa, Artificial sequence
  Chemically synthesized
PQAFEFVGEFFTDVS

SEQ ID NO: 39 - protein, 15 aa, Artificial sequence
  Chemically synthesized
LKVFGNFPKLIMTQV

SEQ ID NO: 40 - protein, 15 aa, Artificial sequence
  Chemically synthesized
LQVTRIFLQALNLGI

SEQ ID NO: 41 - protein, 15 aa, Artificial sequence
  Chemically synthesized
QLMNPGLPDSALDIN

SEQ ID NO: 42 - protein, 15 aa, Artificial sequence
  Chemically synthesized
TQVSKSLQVTRIFLQ

SEQ ID NO: 43 - protein, 15 aa, Artificial sequence
  Chemically synthesized
GRMLTRMWYCSYCQG

SEQ ID NO: 44 - protein, 15 aa, Artificial sequence
  Chemically synthesized
NAGKLTTTIGKLCAH

SEQ ID NO: 45 - protein, 15 aa, Artificial sequence
  Chemically synthesized
TACLVVAMLLSLDFP

SEQ ID NO: 46 - protein, 15 aa, Artificial sequence
  Chemically synthesized
QLLQSASMELKFLII

SEQ ID NO: 47 - protein, 15 aa, Artificial sequence
  Chemically synthesized
LSLEELVNGMYRIYD

SEQ ID NO: 48 - protein, 15 aa, Artificial sequence
  Chemically synthesized
EHEETLSSRRRELIQ

SEQ ID NO: 49 - protein, 15 aa, Artificial sequence
  Chemically synthesized
LGSDINVDDMVNELF

SEQ ID NO: 50 - protein, 15 aa, Artificial sequence
  Chemically synthesized
QVRSFFQRLQPGLKW

SEQ ID NO: 51 - protein, 15 aa, Artificial sequence
  Chemically synthesized
FEIVVRHAKNYTNAM

SEQ ID NO: 52 - protein, 15 aa, Artificial sequence
  Chemically synthesized
HAKNYTNAMFKNNYP

SEQ ID NO: 53 - protein, 15 aa, Artificial sequence
  Chemically synthesized
VGEFFTDVSLYILGS

SEQ ID NO: 54 - protein, 15 aa, Artificial sequence
  Chemically synthesized
IQYVQKNAGKLTTTI

SEQ ID NO: 55 - protein, 15 aa, Artificial sequence
  Chemically synthesized
RQYRSAYYPEDLFID

SEQ ID NO: 56 - protein, 15 aa, Artificial sequence
  Chemically synthesized
KMKGPEPVVSQIIDK

SEQ ID NO: 57 - protein, 15 aa, Artificial sequence
  Chemically synthesized
INQLLRTMSMPKGRV

SEQ ID NO: 58 - protein, 15 aa, Artificial sequence
  Chemically synthesized
WPFLEGCACTPERMA

SEQ ID NO: 59 - protein, 15 aa, Artificial sequence
  Chemically synthesized
MGAPTLPPAWQPFLK

SEQ ID NO: 60 - protein, 15 aa, Artificial sequence
  Chemically synthesized
CACTPERMAEAGFIH

SEQ ID NO: 61 - protein, 15 aa, Artificial sequence
  Chemically synthesized
FLKDHRISTFKNWPF

SEQ ID NO: 62 - protein, 15 aa, Artificial sequence
  Chemically synthesized
ISTFKNWPFLEGCAC

SEQ ID NO: 63 - protein, 15 aa, Artificial sequence
  Chemically synthesized
KHSSGCAFLSVKKQF

SEQ ID NO: 64 - protein, 15 aa, Artificial sequence
  Chemically synthesized
AFLSVKKQFEELTLG

SEQ ID NO: 65 - protein, 15 aa, Artificial sequence
  Chemically synthesized
TLGEFLKLDRERAKN

SEQ ID NO: 66 - protein, 15 aa, Artificial sequence
  Chemically synthesized
AKNKIAKETNNKKKE

SEQ ID NO: 67 - protein, 16 aa, Artificial sequence
  Chemically synthesized
TAKKVRRAIEQLAAMD

SEQ ID NO: 68 - protein, 15 aa, Artificial sequence
  Chemically synthesized
AQCFFCFKELEGWEP

SEQ ID NO: 69 - protein, 15 aa, Artificial sequence
  Chemically synthesized
KLDRERAKNKIAKET

SEQ ID NO: 70 - protein, 15 aa, Artificial sequence
  Chemically synthesized
KQFEELTLGEFLKLD

SEQ ID NO: 71 - protein, 15 aa, Artificial sequence
  Chemically synthesized
PPAWQPFLKDHRIST

SEQ ID NO: 72 - protein, 15 aa, Artificial sequence
  Chemically synthesized
RMAEAGFIHCPTENE

SEQ ID NO: 73 - protein, 15 aa, Artificial sequence
  Chemically synthesized
FIHCPTENEPDLAQC

SEQ ID NO: 74 - protein, 15 aa, Artificial sequence
  Chemically synthesized
ENEPDLAQCFFCFKE

SEQ ID NO: 75 - protein, 15 aa, Artificial sequence
  Chemically synthesized
FKELEGWEPDDDPIE

SEQ ID NO: 76 - protein, 15 aa, Artificial sequence
  Chemically synthesized
WEPDDDPIEEHKKHS

SEQ ID NO: 77 - protein, 15 aa, Artificial sequence
  Chemically synthesized
PIEEHKKHSSGCAFL

SEQ ID NO: 78 - protein, 15 aa, Artificial sequence
  Chemically synthesized
KETNNKKKEFEETAK

SEQ ID NO: 79 - protein, 15 aa, Artificial sequence
  Chemically synthesized
KKEFEETAKKVRRAI

SEQ ID NO: 80 - protein, 10 aa, Artificial sequence
  Chemically synthesized
KIAKETNNKK

SEQ ID NO: 81 - protein, 9 aa, Artificial sequence
  Chemically synthesized
RAIEQLAAM

SEQ ID NO: 82 - protein, 9 aa, Artificial sequence
  Chemically synthesized
LTLGEFLKL

SEQ ID NO: 83 - protein, 9 aa, Artificial sequence
  Chemically synthesized
KVRRAIEQL

SEQ ID NO: 84 - protein, 9 aa, Artificial sequence
  Chemically synthesized
SVKKQFEEL

SEQ ID NO: 85 - protein, 10 aa, Artificial sequence
  Chemically synthesized
QVSKSLQVTR

SEQ ID NO: 86 - protein, 9 aa, Artificial sequence
  Chemically synthesized
EIDKYWREY

SEQ ID NO: 87 - protein, 9 aa, Artificial sequence
  Chemically synthesized
GEFFTDVSL

SEQ ID NO: 88 - protein, 9 aa, Artificial sequence
  Chemically synthesized
LLTSMAISV

SEQ ID NO: 89 - protein, 9 aa, Artificial sequence
  Chemically synthesized
KVFGNFPKL

SEQ ID NO: 90 - protein, 9 aa, Artificial sequence
  Chemically synthesized
FVGEFFTDV

SEQ ID NO: 91 - protein, 9 aa, Artificial sequence
  Chemically synthesized
RLQPGLKWV

SEQ ID NO: 92 - protein, 9 aa, Artificial sequence
  Chemically synthesized
ELFDSLFPV

SEQ ID NO: 93 - protein, 9 aa, Artificial sequence
  Chemically synthesized
RMLTRMWYC

SEQ ID NO: 94 - protein, 10 aa, Artificial sequence
  Chemically synthesized
SLTPQAFEFV

SEQ ID NO: 95 - protein, 8 aa, Artificial sequence
  Chemically synthesized
GILGFVTL